(19) United States
(12) Patent Application Publication
(10) Pub. No.: US 2024/0352419 A1
(43) Pub. Date: Oct. 24, 2024

(54) METHODS AND COMPOSITIONS FOR DIFFERENTIATION OF PLURIPOTENT STEM CELLS AND DERIVED HEMATOPOIETIC LINEAGE CELLS

(71) Applicant: NUWACELL BIOTECHNOLOGIES CO., LTD., Hefei, Anhui (CN)

(72) Inventors: Junying YU, Hefei, Anhui (CN); Tao ZHOU, Hefei, Anhui (CN); Changqing TIAN, Hefei, Anhui (CN); Yali GAO, Hefei, Anhui (CN); Ying ZHANG, Hefei, Anhui (CN)

(73) Assignee: NUWACELL BIOTECHNOLOGIES CO., LTD., Hefei, Anhui (CN)

(21) Appl. No.: 18/025,588

(22) PCT Filed: Dec. 2, 2022
(86) PCT No.: PCT/CN2022/136148

§ 371 Date: Mar. 9, 2023

(51) Int. Cl. C12N 5/0783; C12N 5/071; C12N 5/0789

(52) U.S. Cl. CPC C12N 5/0646; C12N 5/0647; C12N 5/069; C12N 2500/30; C12N 2500/90; C12N 2501/165; C12N 2501/91; C12N 2506/45; C12N 2513/00

(57) ABSTRACT

The present disclosure provides, inter alia, methods and compositions for differentiation of pluripotent stem cells and derived hematopoietic lineage cells including hemogenic endothelial cells, hematopoietic progenitor cells and natural killer cells. The differentiation efficiency for the hemogenic endothelial cells, the hematopoietic progenitor cells and the natural killer cells can be improved by using the methods and compositions of this disclosure described herein.

### TECHNICAL FIELD

The present disclosure generally relates to the field of stem cell technology, in particular to methods and compositions for promoting the directed differentiation of pluripotent stem cells and derived hematopoietic lineage cells.

### BACKGROUND

Natural Killer (NK) cells are innate lymphocytes and play an important role in the defense of, for example, viral infection and cancer, as well as in immune regulation. Due to their innate properties, NK cells have become highly considered for use in immune-based therapies against various diseases and/or disorders. However, there are a plurality of challenges in sourcing NK cells (e.g., for therapeutic uses and biomedical research). Such challenges include, for example, the number of primary NK cells that can be isolated (e.g., during apheresis), the marked variability in the quantity and/or quality of primary NK cells between donors, and/or efficient production of safe NK cells with a well-understood phenotype and/or functionality (e.g., a phenotype and/or functionality similar to primary NK cells) by alternative methods, such as differentiation of pluripotent cells (e.g., hPSCs) into NK cells (iNK cells).

Pluripotent stem cell technology, including human PSC (hPSC) technology, is a highly promising and potentially unlimited source of therapeutically viable cells. However, current protocols of directed differentiation (e.g., to hemogenic endothelial (HE) cells, hematopoietic progenitor (HP) or iNK cells) and subsequent expansion are typically less efficient. In addition, these protocols typically require the use of serums. Use of such components can incur potential risk for contamination, may cause lot dependency in cells, and can be unsuitable for producing cells for clinical and therapeutic use. For example, cells cultured in such xeno-contaminated environments are generally considered unsuitable for use in humans because exposure to animal components may present a serious risk of, for example, immune rejection, transmission of unidentified pathogens to treated subjects, and reactivation of animal retroviruses. The production of high numbers of HPs or iNK cells with a consistently reproducible phenotype and/or functionality (e.g., a phenotype and/or functionality similar to or superior over primary NK cells) also remains a challenge with current methods of directed differentiation.

To advance technologies related to directed differentiation of pluripotent cells (e.g., hPSCs), it is important to be able to efficiently, safely, and/or reproducibly generate not only PSCs and partially differentiated cells (e.g., hematopoietic progenitors), but also immune effector populations, including iNK cells. Accordingly, there remains a need for improved methods and compositions of directed differentiation of pluripotent stem cells (e.g., hPSCs) into hematopoietic lineage cells including HE, HP or iNK cells (e.g., immature iNK cells, functional iNK cells).

### SUMMARY

The present disclosure provides, among other things, methods and compositions for promoting the directed differentiation of pluripotent cells (e.g., hPSCs) or a cell population thereof to hematopoietic lineage cells such as for example, non-pluripotent cells (e.g., iNK cells) or partially differentiated cells, including, for example, HE cells, and/or HP cells. The present disclosure also relates to cell populations, cell lines, and/or clonal cells generated using the methods and compositions described herein.

In a first aspect, the present disclosure relates to a method for promoting the directed differentiation of pluripotent stem cells (PSCs), comprising the steps of: contacting the PSCs with a maintenance culture medium to form embryoid bodies (EBs); contacting the EBs with a first differentiation culture medium, or with the first differentiation culture medium and a second differentiation culture medium sequentially, to form mesodermal cells; contacting the mesodermal cells with a third differentiation culture medium to form hemogenic endothelial (HE) cells; contacting the HE cells with a fourth differentiation culture medium to form hematopoietic progenitor (HP) cells; and contacting the HP cells with a fifth differentiation culture medium to obtain immature iNK cells, wherein a basal medium supplemented with the combination of (i) a nicotinamide-based compound, (ii) a heparin-based compound, and (iii) a human platelet lysate is used as a basal medium of the fourth differentiation culture medium and/or a basal medium of the fifth differentiation culture medium.

In a second aspect, the present disclosure relates to a method for promoting the directed differentiation of pluripotent stem cells (PSCs), comprising the steps of: contacting the PSCs with a maintenance culture medium to form embryoid bodies (EBs); contacting the EBs with a first differentiation culture medium supplemented with a Wnt signaling pathway activator, or with a first differentiation culture medium supplemented with a Wnt signaling pathway activator and a second differentiation culture medium supplemented with a Wnt signaling pathway activator sequentially, to form mesodermal cells; and contacting the mesodermal cells with a third differentiation culture medium supplemented with a Wnt signaling pathway inhibitor to obtain hemogenic endothelial (HE) cells.

In a third aspect, the present disclosure relates to a culture medium for promoting the directed differentiation of pluripotent stem cells (PSCs) into hematopoietic lineage cells, comprising a basal medium and supplemented with a Wnt signaling pathway inhibitor.

In a fourth aspect, the present disclosure relates to a kit comprising the culture medium of the third aspect of the present disclosure.

In a fifth aspect, the present disclosure relates to a method for producing iNK cells, comprising the method for promoting the directed differentiation of pluripotent stem cells (PSCs) according to the first aspect of the present disclosure, and the step for expanding and maturing the immature iNK cells.

In a sixth aspect, the present disclosure relates to a cell population produced by the method according to the first aspect, the second aspect, or the fifth aspect of the present disclosure.

In a seventh aspect, the present disclosure relates to a cell population, wherein more than 90% of the cells in the population without any enrichment or purification are mature CD56+CD3− iNK cells.

In an eighth aspect, the present disclosure relates to a pharmaceutical composition comprising the cell population according to the seventh aspect of the present disclosure and a pharmaceutically acceptable carrier.

In a ninth aspect, the present disclosure relates to use of the cell population according to the seventh aspect of the present disclosure in the manufacture of a medicament for treating or preventing cancer.

Various objects and advantages of the reagents, compositions and methods as provided herein will become apparent from the following description taken in conjunction with the accompanying drawings wherein are set forth, by way of illustration and example, certain embodiments of the present disclosure.

### BRIEF DESCRIPTION OF THE DRAWINGS

FIG. 1 shows a graphic representation of the process for producing iNK cells from hPSCs according to an exemplary example of the method of the present disclosure.

FIG. 2 shows the effect of the concentration of nicotinamide (NAM) in an exemplary basal medium on the expansion of PBNK cells including NK cell expansion fold (FIG. 2A) and the percentage of CD56+CD3− NK cells (FIG. 2B) according to the Example 1 of the present disclosure.

FIG. 3 shows the effect of the concentration of heparin sodium in an exemplary NKSFM basal medium on the expansion of PBNK cells including NK cell expansion fold (FIG. 3A), the percentage of CD56+CD3− NK cells (FIG. 3B) and the lysis of K562 tumor cells (FIG. 3C) according to the Example 2 of the present disclosure.

FIG. 4 shows the effect of the concentration of PLT in an exemplary NKSFM basal medium on the expansion of PBNK cells from donors 1 and 2 including NK cell expansion fold (FIG. 4A), the percentage of CD56+CD3− NK cells (FIG. 4B) and the lysis of K562 tumor cells (FIG. 4C) according to the Example 3 of the present disclosure.

FIG. 5 shows the comparison between the effects of an exemplary NKSFM basal medium and a commercial kit on the expansion of PBNK cells including NK cell expansion fold (FIG. 5A), the percentage of CD56+CD3− NK cells (FIG. 5B) and the lysis of K562 tumor cells (FIG. 5C) according to the Example 4 of the present disclosure, wherein 1 represents NKSFM (feeder-free), 2 represents Commercial kit (feeder-free), 3 represents NKSFM (with feeder), and 4 represents Commercial kit (with feeder).

FIG. 6 shows the effect of different basal medium used during Stage 2 of the present method on the differentiation efficiency for iNK cells (CD56+% (FIG. 6A) and CD56+ cell number (FIG. 6B) according to the Example 6 of the present disclosure, wherein 1, 2, 3, 4, 5, 6 and 7 represent #1, #2, #3, #4, #5, #6 and #7, respectively.

FIG. 7 shows the effect of plate coating during Stage 2 of the present method on iNK differentiation according to the Example 7 of the present disclosure, wherein FIGS. 7A and 7C show a representative morphology and a flow cytometric diagram of cells differentiated by using an exemplary NKSFM basal medium on a cell culture surface without any coating matrix, respectively; and FIGS. 7B and 7D show a representative morphology and a flow cytometric diagram of cells differentiated by using the exemplary NKSFM basal medium on a cell culture surface coated with DLL4 and VCAM1, respectively.

FIG. 8 shows the comparison between the effects of XAV939 and/or SB431542 used during Stage 1-4 of the present method on the differentiation efficiency for HP cells (FIG. 8A) or iNK cells (FIGS. 8B and 8C) according to the Example 8 of the present disclosure, wherein None represents the case where both XAV939 and SB431542 are not used during Stage 1-4, XAV represents the case where XAV939 is used during Stage 1-4, SB represents the case where SB431542 is used during Stage 1-4, and SB+XAV represents the case where both XAV939 and SB431542 are used during Stage 1-4.

FIG. 9 shows the effect of the concentration of CHIR99021 used during Stage 1-2 of the present method on the differentiation efficiency for HP cells (FIG. 9A) or iNK cells (FIGS. 9B and 9C) according to the Example 9 of the present disclosure.

FIG. 10 shows the effect of the concentration of CHIR99021 used during Stage 1-3 of the present method on the differentiation efficiency for HP cells (FIG. 10A) or iNK cells (FIGS. 10B and 10C) according to the Example 10 of the present disclosure, wherein iPSC line 1 represents the case where the hPSC for differentiation is iPSC line 1, and iPSC line 2 represents the case where the hPSC for differentiation is iPSC line 2.

FIG. 11 show the effects of different VEGF concentration in the Stage 1 (Stages 1-2, 1-3, 1-4 and 1-5) of the present method on the differentiation efficiency for the HE cells (KDR+%, FIG. 11A), the HP cells (CD34+%, FIG. 11B) or the iNK cells (output indicated by cell morphology, FIG. 11C) according to the Example 11 of the present disclosure, wherein 1-10 represent #1-10, respectively.

FIG. 12 shows the effect of modulation of VEGF concentration in the Stage 1 (Stages 1-2, 1-3, 1-4 and 1-5) of the present method on the differentiation efficiency for the HE cells (FIG. 12A), the HP cells (FIG. 12B) or the iNK cells (FIGS. 12C and 12D) according to the Example 12 of the present disclosure, wherein 1-4 represent #1-4, respectively.

FIG. 13 shows the effect of IL-10 and/or IL-18 added during Stage 3 of the present method on the specific lysis of K562 tumor cells by iNK cells (FIG. 13A), and the effect of IL-18 added during Stage 3 of the present method on the iNK cell expansion fold according to the Examples 13-14 of the present disclosure, wherein, in FIG. 13A, None represents the case where both IL-10 and IL-18 are not added during Stage 3, IL-10 represents the case where IL-10 is added during Stage 3, IL-18 represents the case where IL-18 is added during Stage 3 and IL-10+IL-18 represents the case where both IL-10 and IL-18 are added during Stage 3; and in FIG. 13B, None represents the case where only IL-2 is added during Stage 3, S3-wk1 represents the case where IL-2 is added during Stage 3 and IL-18 is added in the week 1 of Stage 3 and S3-wk2 represents the case where IL-2 is added during Stage 3 and IL-18 is added in the week 2 of Stage 3.

FIG. 14 shows the morphologies of hematopoietic lineage cells formed during the different stages (D-1, D0, Stage 1-4, Stage 1-5, Stage 2 and Stage 3) of the method of producing iNK cells from hPSCs according to the Example 15 of the present disclosure.

FIG. 15 shows the flow cytometric diagrams of hematopoietic lineage cells formed during the different stages (Stage 1-4, Stage 1-5, Stage 2 and Stage 3) of the method of producing iNK cells from hPSCs according to the Example 15 of the present disclosure.

FIG. 16 shows the long-term expansion potential of the immature iNK cells according to the Example 16 of the present disclosure.

FIG. 17 shows the comparison between the expression of surface receptors NKG2D, NKp30, NKG2A, KIRe1 and CCR6 on the mature iNK cells and on PBNK cells according to the Example 17 of the present disclosure.

FIG. 18 shows the global gene expression profiling of the mature iNK cells, PBNK cells and CBNK cells according to the Example 18 of the present disclosure.

FIG. 19 shows the cytotoxicity of the mature iNK cells against a wide range of tumor cell lines according to the Example 19 of the present disclosure.

FIG. 20 shows the level of pro-inflammatory cytokines secreted by the mature iNK cells according to the Example 20 of the present disclosure.

### DETAILED DESCRIPTION

It is to be appreciated that certain aspects, modes, embodiments, variations and features of the present disclosure are described below in various levels of detail in order to provide a substantial understanding of the present technology.

Reference throughout this specification to “first,” “second,” “third,” “fourth,” “fifth,” “sixth,” “seventh,” “eighth,” or “ninth” does not mean the order or sequence of the feature, structure (e.g., medium or composition) or characteristic described in connection with the reference and can be used only for the purpose of distinction.

Reference throughout this specification to “a first aspect,” “a second aspect,” “a third aspect,” “a fourth aspect,” “a fifth aspect,” “a sixth aspect,” “a seventh aspect,” “an eighth aspect,” or “a ninth aspect” means that a particular feature, structure or characteristic described in connection with the aspect is included in at least one or more aspects of the present disclosure. Also, the particular feature(s), structure(s), characteristic(s) or embodiment(s) in one aspect may be combined with those in one or more other aspects in any suitable manner.

Reference throughout this specification to “one embodiment,” “some embodiments,” “a preferred embodiment(s),” “certain embodiments” or “a certain embodiment(s)” means that a particular feature, structure or characteristic described in connection with the embodiment(s) is included in at least one or more embodiments of the present disclosure. Also, the particular feature(s), structure(s), or characteristic(s) in one embodiment may be combined with those in one or more other embodiments in any suitable manner.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The present disclosure is based, at least in part, on the discovery of a basal medium supplemented with the combination of (i) nicotinamide-based compound, (ii) heparin-based compound, and (iii) human platelet lysate, which can improve the differentiation efficiency for immature NK cells by its stage-specific (after the formation of hemogenic endothelial (HE) cells) use in the process of directed differentiation of pluripotent stem cells (PSCs). The present disclosure is also based, at least in part, on the discovery of a differentiation culture medium supplemented with a Wnt signaling pathway inhibitor, which can, alone or in conjunction with an another differentiation culture medium supplemented with a Wnt signaling pathway activator, improve the differentiation efficiency for HE cells, HP cells or immature NK cells in the process of directed differentiation of pluripotent stem cells (PSCs).

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Unless otherwise specified, “a” or “an” means “one or more.”

As used herein, “about” means plus or minus 10%, or plus or minus 5%, or plus or minus 4%, or plus or minus 3%, or plus or minus 2%, or plus or minus 1%, as well as the specified number.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of). Further, in each instance herein any of the terms “comprising,” “consisting essentially of,” and “consisting of” may be replaced with either of the other two terms.

As used herein, the term “pluripotent stem cell” (PSC) refers to cells derived from the inner cell mass of the embryonic blastocyst. Pluripotent stem cells can be pluripotent and give rise during development to all derivatives of the three primary germ layers: ectoderm, endoderm and mesoderm. Pluripotent stem cells can be of human origin (e.g., human PSC or hPSC). Pluripotent stems cells can be induced pluripotent stem cells (iPSCs) or embryonic stem cells (ESCs). ESCs (e.g., hESCs) and iPSCs (e.g., hiPSCs) are known in the art and can be readily obtained using conventional methods, for example, those described in the existing technologies, or commercially available products. Suitable methods for the generation of iPSCs from somatic or multipotent stem cells are well known to those of skill in the art. For example, iPSCs may be reliably generated from somatic cells by conventional reprogramming technologies.

As used herein, the term “pluripotency” or “pluripotent” refers to a cell that has the developmental potential to differentiate into cells of all three germ layers (Ectoderm, mesoderm, and endoderm). Pluripotency can be determined, at least in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “pluripotent stem cell morphology” refers to the classical morphological features of an embryonic stem cell. Normal embryonic stem cell morphology can be characterized as small and round in shape, with a high nucleus-to-cytoplasm ratio, the notable presence of nucleoli, and/or typical inter-cell spacing.

As used herein, the term “reprogramming” refer to a method of increasing the potency of a cell or dedifferentiating a cell to a less differentiated state. For example, a cell that has an increased cell potency can have more developmental plasticity (i.e., can differentiate into more cell types) compared to the same cell in the non-reprogrammed state. That is, a reprogrammed cell is one that is in a less differentiated state than the same cell in a non-reprogrammed state. “Reprogramming” can refer to de-differentiating a somatic cell, or a multipotent stem cell, into a pluripotent stem cell, also referred to as an induced pluripotent stem cell, or iPSC.

As used herein, the term “differentiation” refers to the process by which an unspecialized (“uncommitted”) or less specialized cell acquires the features of a specialized cell such as, for example, a blood cell or an immune cell. In certain embodiments, a differentiated or differentiation-induced cell is one that has taken on a more specialized (“committed”) position within the lineage of a cell. For example, a human Pluripotent Stem Cell (hPSCs) can be differentiated into various more differentiated cell types, for example, a neural or a hematopoietic progenitor cell, a lymphocyte, a cardiomyocyte, an immune cell (e.g., a Natural Killer cell), and other cell types, upon treatment with suitable differentiation factors in the cell culture medium. In certain embodiments, the term “committed” is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

As used herein, the term “hematopoietic lineage cells” refers to cells differentiated in vitro from PSCs and/or their progeny and may include one or more of the following: hemangioblasts, hemogenic endothelial cells (HECs), hematopoietic stem cells, hematopoietic progenitor cells (HPCs), erythroid/megakaryocytic progenitor cells, erythrocytes, megakaryocytes, platelets, and lymphoid lineage cells.

As used herein, the term “lymphoid lineage cells” includes one or more of: lymphoid progenitor cells, lymphocytes (such as T lymphocytes), natural killer (NK) cells, myeloid progenitor cells, granulomonocytic progenitor cells, monocytes, macrophages, and dendritic cells.

As used herein, the term “embryoid body” (EB) refers to a three-dimensional cluster that have been shown to mimic embryo development as it gives rise to numerous lineages within its three-dimensional area.

As used herein, the term “mesoderm” or “mesodermal cells” refers to one of the three germ layers or cells therefrom that appears during early embryogenesis and which gives rise to various specialized cell types including blood cells of the circulatory system, muscles, the heart, the dermis, skeleton, and other supportive and connective tissues.

As used herein, the term “hemogenic endothelium” (HE), “hemogenic endothelium cell” or “hemogenic endothelial cell” refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

As used herein, the term “hematopoietic progenitor” (HP) or “hematopoietic progenitor cell” (HPC) refers to cells present in the blood and bone marrow capable of forming mature blood cells, such as red blood cells, platelets, and immune cells.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

As used herein the term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In certain embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, “natural killer cells” or NK cells refers to lymphoid cells defined by its marker expression and function/activity. For example, in humans, NK cells expresses CD56. For example, such NK cells may be CD56+CD3-cells. NK cells may express variable levels of CD56. NK cells may comprise primary NK cells or induced NK (INK) cells.

As used herein, “primary NK cells” refers to naturally occurring natural killer cells which can be sourced from, for example, blood (e.g., cord blood or peripheral blood collected by apheresis), bone marrow or frozen primary NK cells (e.g., commercially available). Examples of primary NK cells comprises PBNK (peripheral blood-derived NK) and CBNK (cord blood-derived NK) cells.

As used herein, the term “INK cells” refers to natural killer cells differentiated from pluripotent stem cells (e.g., hPSCs) and expanded and matured. The iNK cells may be, for example, iPSC-derived iNK cells or ESC-derived iNK cells. The iNK cells can be used interchangeably with mature iNK cells. The mature iNK cells have higher expression level for specific markers such as CD56 over immature iNK cells and have the cytokine-releasing function and cytotoxity similarly to primary NK cells.

As used herein, the term “immature iNK cells” refers to natural killer cells differentiated from pluripotent cells (e.g., hPSCs) and not expanded and matured. The immature iNK cells may be, for example, iPSC-derived immature iNK cells or ESC-derived immature iNK cells. The immature iNK cells have lower expression level for specific markers such as CD56 and have lower cytokine-releasing function and cytotoxity as compared with mature iNK cells.

As used herein, the term “culture medium” refers to a culture medium which can support the survival, growth, propagation, maintenance and/or differentiation of cells in an in vitro environment. A culture medium may have a basal medium and one or more supplements.

As used herein, the term “maintenance culture medium” refers to a culture medium which can support the survival, growth, propagation, or maintenance of cells in an in vitro environment.

As used herein, the term “differentiation culture medium” or “differentiation culture media” refers to a culture medium(s) which can support the differentiation of cells in an in vitro environment.

As used herein, the term “basal medium” refers to a basal component of a culture medium (e.g. differentiation culture medium, or expansion culture medium) relative to its supplement(s). Generally, the basal medium comprises about 95% to 99% by volume of the culture medium (e.g. differentiation culture medium, or expansion culture medium). A basal medium of a cell maintenance culture medium acts as a source of nutrients, hormones and/or other factors helpful to propagate and/or sustain the cells. A basal medium of a cell differentiation culture medium acts as a source of nutrients, hormones and/or other factors helpful to differentiate the cells.

As used herein, the term “supplement(s)” refers to an additive component(s) of a culture medium (e.g. differentiation culture medium, or expansion culture medium) relative to its basal medium.

As used herein, the term “supplemented” refers to the addition of a supplement for a culture medium (e.g. differentiation culture medium) into its basal medium. The supplement(s) may be added into a basal medium of a culture medium before or upon the use of the culture medium.

As used herein, the term “in vitro” refers generally to activities that take place outside an organism.

As used herein, the term “in vivo” refers generally to activities that take place inside an organism.

As used herein, the term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments this term can be used interchangeably with ex vivo.

As used herein, the terms “feeder cells” or “feeders” refer to cells of one type that are co-cultured with cells of a second type to provide an environment in which the cells of the second type can grow, as the feeder cells provide growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. The feeder cells may typically be inactivated when being co-cultured with other cells by irradiation or treatment with an anti-mitotic agent such as mitomycin to prevent them from outgrowing the cells they are supporting. Feeder cells may include, for example, endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage and promote maturation to a specialized cell types, such as an effector cell.

As used herein, a “feeder-free” (FF) environment refers to an environment such as a culture condition, cell culture or culture media which is essentially free of feeder or stromal cells, and/or which has not been pre-conditioned by the culture of feeder cells.

As used herein, the term “cell population” or “population of cells” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “effective amount” refers to a quantity of an agent sufficient to achieve a beneficial or desired result upon administration. The amount of an agent administered to the subject can depend on the characteristics of the individual, such as general health, age, sex, body weight, effective concentration of the cells (e.g., iNK cells) administered, and tolerance to drugs. The skilled artisan will be able to determine appropriate dosages depending on these and other factors. An effective amount can be administered to a subject in one or more doses.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular medical intervention, or from whom cells are harvested). In certain embodiments, the individual, patient or subject is a human.

As used herein, the terms “treatment,” “treat,” and “treating” refer to a clinical intervention aimed to reverse, alleviate, delay the onset of, or inhibit the progress, ameliorate, reduce severity of, prevent or delay the recurrence of a disease, disorder, and/or condition or one or more symptoms thereof, and/or improve one or more symptoms of a disease, disorder, and/or condition as described herein. Treatment, e.g., in the form of an iNK cell or a population of iNK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence. Treatment can result in improvement and/or resolution of one or more symptoms of a disease, disorder and/or condition.

As used herein, the terms “prevent,” “preventing,” and “prevention” refer to reducing the probability of developing a disease, disorder, or condition in a subject, who does not have, but is at risk of or susceptible to developing a disease, disorder, or condition.

### First Differentiation Method

The first aspect of the present disclosure relates to a method for promoting the directed differentiation (e.g., hematopoietic differentiation) of pluripotent stem cells (PSCs) (into e.g., iNK cells), comprising the steps of: contacting the PSCs with a maintenance culture medium to form embryoid bodies (EBs); contacting the EBs with a first differentiation culture medium, or with the first differentiation culture medium and a second differentiation culture medium sequentially, to form mesodermal cells; contacting the mesodermal cells with a third differentiation culture medium to form hemogenic endothelial (HE) cells; contacting the HE cells with a fourth differentiation culture medium to form hematopoietic progenitor (HP) cells; and contacting the HP cells with a fifth differentiation culture medium to obtain immature iNK cells, wherein a basal medium supplemented with the combination of (i) a nicotinamide-based compound, (ii) a heparin-based compound, and (iii) a human platelet lysate is used as a basal medium of the fourth differentiation culture medium and/or a basal medium of the fifth differentiation culture medium.

According to the first aspect of the present disclosure, due to the stage-specific (after the formation of hemogenic endothelial (HE) cells) use of a basal medium supplemented with the combination of (i) nicotinamide-based compound, (ii) heparin-based compound, and (iii) human platelet lysate (hereinafter referred to as a SFM basal medium), the above method can improve the differentiation efficiency for immature iNK (CD56+) cells, or for HP cells and immature iNK (CD56+) cells. The above method may be accomplished within about 20-40 days and produce a high number of immature iNK cells. For example, 3×10⁸ immature iNK cells can be derived from 1× 10⁶ hPSCs.

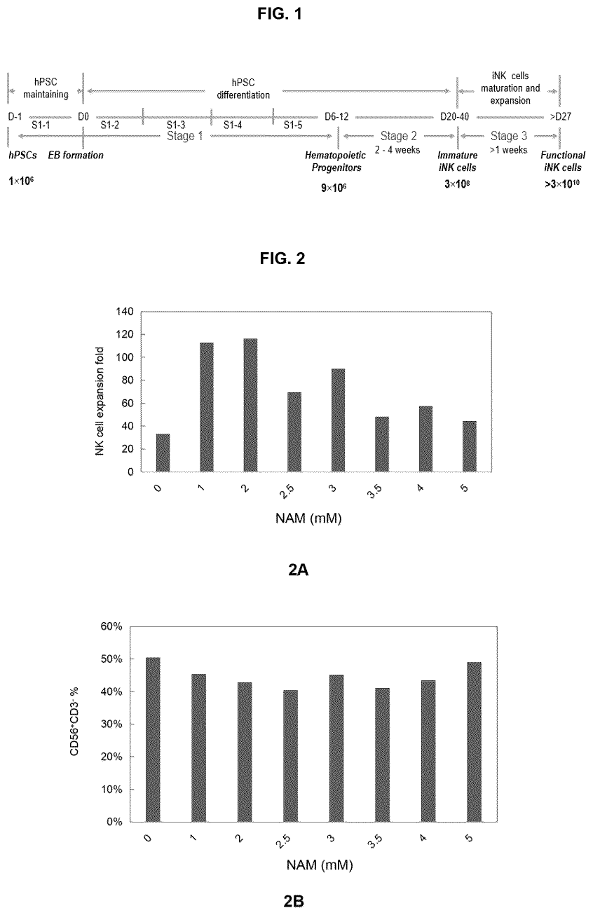

FIG. 1 shows a graphic representation of the process for producing iNK cells from hPSCs according to an exemplary example of the method of the present disclosure. As shown in FIG. 1, hPSCs are firstly differentiated into hematopoietic progenitors (Stage 1), and then further differentiated into immature NK cells (Stage 2). The Stage 1 can be further divided into 5 substages, that is, Stages 1-1, 1-2, 1-3, 1-4 and 1-5. The Stage 1-1 is a hPSC maintaining stage. The Stages 1-2 and 1-3 are substages of hPSCs differentiation into mesodermal cells, the Stages 1-4 is a substage of mesoderm differentiation into HE cells, and the Stage 1-5 is a substage of HE differentiation into HP cells. The Stage 3 is a stage of iNK cells expansion and maturation, and will be described hereinafter.

According to the present disclosure, the SFM basal medium may be obtained by adding (i) nicotinamide-based compound, (ii) heparin-based compound, and (iii) human platelet lysate into a common basal medium in the art or any other suitable basal medium such as IF-4 basal medium or CD34A basal medium (e.g., those used in the examples). The examples of the common basal medium may comprise IMDM/F12, Ham's F12, IMDM, BME, DMEM, RPMI-1640, α-MEM, all of which are commercially available, and any combination thereof.

In certain embodiments, the SFM basal medium is continuously used throughout the step of contacting the HE cells with the fourth differentiation culture medium to form the HP cells, and the SFM basal medium is discontinuously used throughout the step of contacting the HP cells with the fifth differentiation culture medium to obtain the immature iNK cells.

In certain embodiments, the SFM basal medium and an another different basal medium are individually used at an arbitrary order as the basal medium of the fifth differentiation culture medium throughout the step of contacting the HP cells with the fifth differentiation culture medium to obtain the immature iNK cells.

In preferred embodiments, the SFM basal medium is continuously used throughout the step of contacting the HE cells with the fourth differentiation culture medium to form the HP cells and throughout the step of contacting the HP cells with the fifth differentiation culture medium to obtain the immature iNK cells. According to the above embodiments, the differentiation efficiencies for HP cells and immature iNK (CD56+) cells can be further improved.

In certain embodiments, the first to third differentiation basal media comprise different basal media.

In certain embodiments, the first to third differentiation basal media comprise the same basal medium, which is helpful to simplify the differentiation protocol of the present disclosure.

In case where the SFM basal medium and an another different basal medium are individually used in the step of contacting the HP cells with the fifth differentiation culture medium to obtain the immature iNK cells, the another different basal medium may be same as, similar to, or different from those in the first to third differentiation culture media. In preferred embodiments, a basal medium supplemented with only one or two of (i) nicotinamide-based compound, (ii) heparin-based compound and (iii) human platelet lysate is used as the another different basal medium. In more preferred embodiments, a basal medium supplemented with (i) nicotinamide-based compound, and only one of (ii) heparin-based compound and (iii) human platelet lysate is used as the another different basal medium. In a most preferred embodiment, a basal medium supplemented with (i) nicotinamide-based compound and (ii) human platelet lysate and without (iii) heparin-based compound is used as the another different basal medium.

In preferred embodiments, the SFM basal medium is continuously used as one of the basal medium of the fourth differentiation culture medium and the basal medium of the fifth differentiation culture medium, and a basal medium supplemented with (i) nicotinamide-based compound and (ii) heparin-based compound and without (iii) human platelet lysate is continuously used as the other of the basal medium of the fourth differentiation culture medium and the basal medium of the fifth differentiation culture medium.

The nicotinamide-based compound, the heparin-based compound and the human platelet lysate will be described in detail below.

### Nicotinamide-Based Compound

As used herein, “nicotinamide-based compound” refers to nicotinamide as well as to analogs thereof and metabolites of nicotinamide or nicotinamide analogs, such as, for example, NAD, NADH and NADPH, and products that are derived from these compounds.

According to embodiments of the present disclosure, the nicotinamide-based compound may be selected from the group consisting of nicotinamide, a nicotinamide analog, a nicotinamide metabolite, a nicotinamide analog metabolite, and derivatives thereof.

Nicotinamide is the amide form of niacin, both of which belong to the vitamin B3 family. They are the precursors of nicotinamide adenine dinucleotide (NAD), which acts as a coenzyme in multiple cellular processes, including energy metabolism and DNA repair. Nicotinamide can be converted into nicotinamide mononucleotide (NMN) by nicotinamide phosphoribosyltransferase (NAMPT), which is then turned into NAD⁺ by nicotinamide mononucleotide adenylyltransferase (NMNAT).

As used herein, “nicotinamide analog” refers to any molecule that is known to act similarly to nicotinamide. Examples of nicotinamide analogs include, without limitation, nicotinethioamides (the thiol analog of nicotinamide), and nicotinic acid. Examples of nicotinamide derivatives include, but are not limited to, substituted nicotinamide-based compound and nicotinethioamides, and N-substituted nicotinamide-based compound and nicotinethioamides.

### Heparin-Based Compound

As used herein, “heparin-based compound” refers to heparain, a derivative thereof, or a salt thereof.

According to embodiments of the present disclosure, the heparin-based compound may be selected from the group consisting of heparin, a derivative thereof, or a salt thereof.

Heparin, a highly sulfated heparin glycosaminoglycan variant produced and stored primarily by mast cells, is understood to possess the highest net negative charge density of all known biological molecules. Its negative charge binds to positively charged heparin-binding domains present in a large number of extracellular proteins. Such proteins include, for example, fibroblast growth factors (FGFs), vascular endothelial growth factors (VEGFs), bone morphogenetic proteins (BMPs), and large extracellular structural molecules such as fibronectin and laminin.

Examples of the derivative of heparin include, without limitation, substituted heparin. Examples of the salt of heparin or a derivative thereof include, without limitation, heparin sodium salt and heparin lithium salt, and the salts of substituted heparin.

### Human Platelet Lysate (PLT)

PLT used according to the present disclosure is commercially available. Human platelet lysate (PLT) may be derived from healthy donor human platelets and is growth factor-rich.

According to the present disclosure, the concentration of the nicotinamide-based compound in the composition of the present disclosure (e.g., the SFM basal medium, or the basal media supplemented with only one or two of (i) nicotinamide-based compound, (ii) heparin-based compound and (iii) human platelet lysate), is not particularly limited. In some embodiments, the concentration of the nicotinamide-based compound in the composition of the present disclosure (e.g., the SFM basal medium, or the basal media supplemented with only one or two of (i) nicotinamide-based compound, (ii) heparin-based compound and (iii) human platelet lysate), is from about 0.5 to about 20 mM, preferably about 1 to about 10 mM, and more preferably about 1 to about 5 mM.

In certain embodiments, the composition of the present disclosure (e.g., the SFM basal medium, or the basal media supplemented with only one or two of (i) nicotinamide-based compound, (ii) heparin-based compound and (iii) human platelet lysate), may comprise the nicotinamide-based compound at a concentration of about 0.1 mM or more, 0.2 mM or more, 0.5 mM or more, 1 mM or more, 2 mM or more, 3 mM or more, 4 mM or more, 5 mM or more, 6 mM or more, 7 mM or more, 8 mM or more, 9 mM or more, 10 mM or more.

According to the present disclosure, the concentration of the heparin-based compound in the composition of the present disclosure (e.g., the SFM basal medium, or the basal media supplemented with only one or two of (i) nicotinamide-based compound, (ii) heparin-based compound and (iii) human platelet lysate), is not particularly limited. In some embodiments, the concentration of the heparin-based compound in the composition of the present disclosure (e.g., the SFM basal medium, or the basal media supplemented with only one or two of (i) nicotinamide-based compound, (ii) heparin-based compound and (iii) human platelet lysate), is preferably from about 0.1 to about 100 μg/mL, and more preferably about 0.5 to about 50 μg/mL.

In certain embodiments, the composition of the present disclosure (e.g., the SFM basal medium, or the basal media supplemented with only one or two of (i) nicotinamide-based compound, (ii) heparin-based compound and (iii) human platelet lysate), may comprise the heparin-based compound at a concentration of about 0.1 μg/ml or more, 0.2 μg/ml or more, 0.3 μg/ml or more, 0.4 μg/ml or more, 0.5 μg/ml or more, 1 μg/ml or more, 2 μg/ml or more, 3 μg/ml or more, 4 μg/ml or more, 5 μg/ml or more, 6 μg/ml or more, 7 μg/ml or more, 8 μg/ml or more, 9 μg/ml or more, 10 μg/ml or more, 20 μg/ml or more, 30 μg/ml or more, 40 μg/ml or more, 50 μg/ml or more, or 100 μg/ml or more.

According to the present disclosure, the concentration of the human platelet lysate in the composition of the present disclosure (e.g., the SFM basal medium, or the basal media supplemented with only one or two of (i) nicotinamide-based compound, (ii) heparin-based compound and (iii) human platelet lysate), is not particularly limited. In some embodiments, the concentration of the human platelet lysate in the composition of the present disclosure (e.g., the SFM basal medium, or the basal media supplemented with only one or two of (i) nicotinamide-based compound, (ii) heparin-based compound and (iii) human platelet lysate), is from about 0.1% to about 20% by volume, preferably about 0.1% to about 10% by volume, and more preferably about 0.1% to about 5% by volume.

In certain embodiments, the composition of the present disclosure (e.g., the SFM basal medium, or the basal media supplemented with only one or two of (i) nicotinamide-based compound, (ii) heparin-based compound and (iii) human platelet lysate), may comprise PLT at a percentage (v/v) of about 0.1%, 0.2%, 0.3%, 0.4%, 0.5%, 1%, 1.5%, 2%, 2.5%, 3%, 3.5%, 4%, 4.5%, 5%, 6%, 7%, 8%, 9%, 10%, or more.

In case where the nicotinamide-based compound, the heparin-based compound or PLT is present in the basal medium (such as the basal media supplemented with only one or two of (i) nicotinamide-based compound, (ii) heparin-based compound and (iii) human platelet lysate) other than the SFM basal medium, the Examples of the nicotinamide-based compound, the heparin-based compound and PLT and their concentrations in the media are same as those in the SFM basal medium.

Optionally, the SFM basal medium may comprise glutamine or its derivative. The examples of the glutamine or its derivative may comprise glutamine, GlutaMAX-1, L-glutamine, and L-alanyl-L-glutamine.

In certain embodiments, glutamine or its derivative may be present in the SFM basal medium at a concentration of about 0.1 to about 5% (v/v). In certain embodiments, glutamine or its derivative may be present in a concentration of about 0.5% (v/v), about 1% (v/v), about 2% (v/v), about 3% (v/v), about 4% (v/v) or about 5% (v/v).

Optionally, the SFM basal medium may comprise one or more antioxidants. The examples of the antioxidant may comprise ascorbic acid or its salt or derivative such as ascorbic acid magnesium salt, ascorbic acid sodium salt, ascorbyl glucoside, 3-ethylascorbic acid, ascorbyl tetraisopalmitate, ascorbic acid phosphate salt, and ascorbyl palmitate.

In certain embodiments, ascorbic acid or its salt or derivative may be present in the SFM basal medium at a concentration of about 10 to about 200 μg/mL, and preferably about 50 to about 150 μg/mL. In certain embodiments, ascorbic acid or its salt or derivative may be present in a concentration of about 20 g/mL, about 40 g/mL, about 60 g/mL, about 80 μg/mL or about 100 μg/mL.

Optionally, the SFM basal medium may comprise Human serum albumin (HSA). In certain embodiments, HSA may be present in the SFM basal medium at a concentration of about 0.1 to about 20 mg/mL. In certain embodiments, HSA may be present in a concentration of about 1 mg/mL, about 5 mg/mL, about 10 mg/mL, about 15 mg/mL or about 20 mg/mL.

Optionally, the SFM basal medium may comprise Monothioglycerol (MTG). In certain embodiments, MTG may be present in the SFM basal medium at a concentration of about 1 to about 400 μM. In certain embodiments, MTG may be present in a concentration of about 10 μM, about 30 μM, about 50 μM, about 70 μM, about 90 μM, about 120 M, about 150 μM or about 200u M.

Optionally, the SFM basal medium may comprise a transferrin. In certain embodiments, transferrin may be present in the SFM basal medium at a concentration of in a concentration of about 1 to about 200 μg/mL, and preferably about 50 to about 150 μg/mL. In certain embodiments, transferrin may be present in a concentration of about 20 μg/mL, about 40 μg/mL, about 60 μg/mL, about 80 μg/mL or about 100 μg/mL.

Optionally, the SFM basal medium may comprise a selenite such as Na selenite. In certain embodiments, selenite may be present in the SFM basal medium at a concentration of in a concentration of about 1 to about 50 ng/mL, and preferably about 5 to about 40 ng/mL. In certain embodiments, selenite may be present in a concentration of about 5 ng/ml, about 10 ng/mL, about 15 ng/ml, about 20 ng/ml or about 30 ng/mL.

Optionally, the SFM basal medium may comprise ethanolamine. In certain embodiments, ethanolamine may be present in the SFM basal medium at a concentration of in a concentration of about 1 to about 100 μM, and preferably about 5 to about 50 μM. In certain embodiments, ethanolamine may be present in a concentration of about 5 μM, about 10 μM, about 20 μM, about 30 μM, about 40 μM, about 60 M, about 80 μM or about 100 μM.

Optionally, the SFM basal medium may comprise pyruvate such as sodium pyruvate. In certain embodiments, pyruvate may be present in the SFM basal medium at a concentration of in a concentration of about 10 to about 500 μg/mL, and preferably about 50 to about 200 μg/mL. In certain embodiments, pyruvate may be present in a concentration of about 20 μg/mL, about 40 μg/mL, about 60 μg/mL, about 80 μg/mL or about 100 μg/mL.

Optionally, the SFM basal medium may comprise an insulin. In certain embodiments, insulin may be present in the SFM basal medium at a concentration of in a concentration of about 0.1 to about 20 μg/mL. In certain embodiments, insulin may be present in a concentration of about 1 μg/mL, about 3 μg/mL, about 5 μg/mL, about 8 μg/mL, about 12 μg/mL, or about 15 μg/mL.

Besides the combination of (i) a nicotinamide-based compound, (ii) a heparin-based compound, and (iii) a human platelet lysate, the basal media of the first to third differentiation culture media and the another different basal medium may comprise the components same as the above optional components in the SFM basal medium. In certain embodiments, the concentrations of these components in the basal media of the first to third differentiation culture media and the another different basal medium may be same as those in the SFM basal medium.

In certain embodiments, the basal media of the first to third differentiation culture media and/or the another different basal medium may be IF-4 or CD34A basal medium, and preferably IF-4 basal medium.

In certain embodiments, the nicotinamide-based compound comprises nicotinamide, and the heparin-based compound comprises heparin sodium. In preferable embodiments, the SFM basal medium contains IF-4 basal medium in addition to the combination of (i) a nicotinamide-based compound, (ii) a heparin-based compound, and (iii) a human platelet lysate. In more preferable embodiments, the SFM basal medium contains IF-4 basal medium in addition to the combination of (i) nicotinamide, (ii) heparin sodium, and (iii) human platelet lysate. In a most preferable embodiment, the SFM basal medium comprises a NKSFM basal medium.

In certain embodiments, the another different basal medium may be NKM or NKSFM-EP basal medium, and preferably NKSFM-EP basal medium.

The SFM basal medium, the basal media of the first to third differentiation culture media and the another different basal medium may also comprise other components such as mercaptoethanol, non-essential amino acid (NEAA), Bovine serum albumin (BSA), sulfate, nitrate, trace elements, CD Lipid Concentrate, and Human serum. The concentrations of these components may be easily determined. In certain embodiments, mercaptoethanol may be contained in these basal media at a concentration of 1 to 100 μM. In certain embodiments, NEAA may be contained in these basal media at a concentration of 0.1 to 5% (v/v). In certain embodiments, BSA may be contained in these basal media at a concentration of 0.1 to 20 mg/mL. In certain embodiments, sulfate may be contained in these basal media at a concentration of 0.1 to 10 ng/mL. In certain embodiments, nitrate may be contained in these basal media at a concentration of 0.1 to 10 μg/mL. In certain embodiments, trace elements may be contained in these basal media at a concentration of 0 to 1% (v/v). In certain embodiments, CD Lipid Concentrate may be contained in these basal media at a concentration of 0 to 1% (v/v). In certain embodiments, human serum may be contained in these basal media at a concentration of 1 to 20% (v/v).

In certain embodiments, a cell culture surface may be coated with a coating matrix. In certain embodiments, a cell culture surface may be not coated with any coating matrix. In preferred embodiments, the method further comprises seeding the HP cells on a cell culture surface coated with a Notch pathway activator and an adhesion molecule. According to the above embodiments, the differentiation efficiency for immature iNK (CD56+) cells can be further improved as compared with the embodiments wherein the cell culture surface is not coated with any coating matrix.

In more preferred embodiments, the Notch pathway activator is selected from DLL4, DLL1, Jagged-1, Jagged-2, variants thereof and any combination thereof, and the adhesion molecule is selected from VCAM1, Fibronectin, Laminin, Vitronectin, MAdCAM-1, ICAM, variants thereof and any combination thereof. In a most preferred embodiment, the cell culture surface is coated with DLL4 and VCAM1. The technology for coating a cell culture surface is conventional in the art and can be easily determined by one skilled in the art.

In certain embodiments, the third differentiation culture medium is further supplemented with a Wnt signaling pathway inhibitor. According to the above embodiments, the differentiation efficiencies for HP cells and immature NK cells can be further improved.

### Wnt Signaling Pathway Inhibitor

The third differentiation culture medium of the present disclosure can utilize a Wnt signaling pathway inhibitor as a supplement. A Wnt signaling pathway inhibitor refers to antagonist of the Wnt signaling pathway (e.g., agents capable of downregulating activity and/or amount of a component participating in the Wnt signaling pathway).

Wnt signaling pathway inhibitors can include, for example, an agent that antagonizes one or more human FZD proteins, an FZD-binding agent. The FZD-binding agent may be an antibody or a polypeptide.

Examples of Wnt signaling pathway inhibitors include, without limitation, one or more of the following: a polypeptide comprising an amino acid sequence of a Wnt antagonist, a small organic molecule that inhibits Wnt/β-catenin signaling, a small organic molecule that inhibits the expression or activity of a Wnt agonist, an antibody that binds to and inhibits the activity of a Wnt agonist and preferably a small organic molecule that inhibits Wnt/β-catenin signaling, and a small organic molecule that inhibits the expression or activity of a Wnt agonist.

Non-limiting examples of Wnt signaling pathway inhibitors further preferably include one or more of the following: iCRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, XAV-939, Foscenvivint (ICG-001), Capmatinib, Isoquercitrin, Gigantol, JW55, MSAB, IWR-1-endo, KY02111, FH535, WIKI4, CCT251545, Prodigiosin, KYA1797K, NCB-0846, LF3, iCRT14, Adavivint, Triptonide, M435-1279, and XAV939, more preferably CRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, Isoquercitrin, Gigantol, JW55, MSAB, IWR-1-endo, FH535, WIKI4, CCT251545, KYA1797K, NCB-0846, iCRT14, Adavivint, M435-1279, and XAV939, and most preferably XAV939.

According to the present disclosure, the concentration of the Wnt signaling pathway inhibitor in the third differentiation culture medium is not particularly limited. In preferred embodiments, the concentration of the Wnt signaling pathway inhibitor in the third differentiation culture medium is from 1 to 30 μM.

In certain embodiments, the third differentiation culture medium of the present disclosure comprises a Wnt signaling pathway inhibitor at a concentration of about 0.1 μM or more, about 0.5 μM or more, such as about 1-30 μM, preferably about 1-20 μM, more preferably 1-10 UM, and most preferably 2-8 μM.

In certain embodiments, the third differentiation culture medium does not contain a TGF-β signaling pathway inhibitor as a supplement.

In case where the third differentiation culture medium is further supplemented with a Wnt signaling pathway inhibitor, the EBs may be contacted only with a first differentiation culture medium further supplemented with a Wnt signaling pathway activator. According to the above embodiments, by fine-tuning (firstly activating and then suppressing the Wnt signaling of cells, or down-regulating the Wnt signaling stepwise) the Wnt signaling during hematopoietic differentiation, the differentiation efficiencies for HP cells and immature NK cells can be further improved.

In case where the third differentiation culture medium is further supplemented with a Wnt signaling pathway inhibitor, the EBs may be contacted with a first differentiation culture medium further supplemented with a Wnt signaling pathway activator and a second differentiation culture medium further supplemented with a Wnt signaling pathway activator sequentially, wherein the Wnt signaling pathway activator in the second differentiation culture medium may be same or different and has an equal or lower concentration as compared to the Wnt signaling pathway activator in the first differentiation culture medium.

In preferred embodiments, the second differentiation culture medium has the same composition as that of the first differentiation culture medium except that the concentration of the Wnt signaling pathway activator in the second differentiation culture medium is lower than the concentration of the Wnt signaling pathway activator in the first differentiation culture medium. According to the above embodiments, due to the smoother transition from the activation to the suppression of Wnt signaling, the differentiation into HP (CD34+) cells and iNK (CD56+) cells can be further facilitated.

According to the present disclosure, the concentrations of the Wnt signaling pathway activator in the first and second differentiation culture media are not particularly limited. In certain embodiments, the first differentiation culture medium or second differentiation culture medium of the present disclosure comprises a Wnt signaling pathway activator at a concentration of 0.5 μM or more, 1 μM or more, 1.5 μM or more, 2 μM or more, 2.5 UM or more, 3 μM or more, 3.5 μM or more, 4 μM or more, 4.5 μM or more, 5 UM or more, 5.5 μM or more, 6 μM or more, 6.5 μM or more, 7 μM or more, 7.5 μM or more, 8 μM or more, 8.5 μM or more, 9 μM or more, 9.5 μM or more, or 10 UM or more. In certain embodiments, the first differentiation culture medium or second differentiation culture medium of the present disclosure comprises a Wnt signaling pathway activator at a concentration of about 1 to 20 μM, preferably about 1 to 10 μM, or more preferably about 1 to 5 μM.

In certain embodiments, the concentration of the Wnt signaling pathway activator in the second differentiation culture medium is at least 50% lower (including, e.g., 55% lower, 60% lower, 65% lower, 70% lower, 75% lower, 80% lower, 85% lower, 90% lower, 95% lower, 99% lower, or 100% lower) than the concentration of the Wnt signaling pathway activator in the first differentiation culture medium.

In preferred embodiments, the concentration of the Wnt signaling pathway activator in the second differentiation culture medium is from 0 to 4 μM, and the concentration of the Wnt signaling pathway activator in the first differentiation culture medium is from 4 to 8 μM. In more preferred embodiments, the concentration of the Wnt signaling pathway activator in the second differentiation culture medium is from 0 to 4 μM, and the concentration of the Wnt signaling pathway activator in the first differentiation culture medium is from 5 to 8 μM.

### Wnt Signaling Pathway Activators

Technologies of the present disclosure can utilize Wnt signaling pathway activators as a supplement.

Wnt signaling pathway activators refers to an agonist of the Wnt signaling pathway (e.g., agents capable of upregulating activity and/or amount of a component participating in the Wnt signaling pathway).

Non-limiting examples of Wnt signaling pathway activators include one or more of the following: a polypeptide comprising an amino acid sequence of a Wnt polypeptide, a polypeptide comprising an amino acid sequence of an activated Wnt receptor, a small organic molecule that promotes Wnt/β-catenin signaling, a small organic molecule that inhibits the expression or activity of a Wnt antagonist, an antibody that binds to and inhibits the activity of a Wnt antagonist, a polypeptide comprising an amino acid sequence of a β-catenin polypeptide, and a polypeptide comprising an amino acid sequence of a Lef-1 polypeptide, and preferably a small organic molecule that promotes Wnt/β-catenin signaling and a small organic molecule that inhibits the expression or activity of a Wnt antagonist.

Wnt signaling pathway activators further include GSK3 inhibitors. GSK3 inhibitors may include, for example and without limitation, polynucleotides, polypeptides, and small molecules. Exemplary GSK3 inhibitors include, for example and without limitation, Kenpaullone, 1-Azakenpaullone, CHIR99021, CHIR98014, NP031112, TWS119, AZD2858, AZD1080, SB415286, LY2090314, AR-A014418, CT 99021, CT 20026, SB216763, AR-A014418, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(1-pyridyl) [1,3,4]-oxadiazole, OTDZT, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, TWS119 pyrrolopyrimidine compound, L803 H-KEAPPAPPQSpP-NH2 or its myristoylated form, 2-Chloro-1-(4,5-dibromo-thiophen-2-yl)-ethanone, GF109203X, RO318220, TDZD-8, TIBPO, and OTDZT, preferably Kenpaullone, 1-Azakenpaullone, CHIR99021, CHIR98014, NP031112, TWS119, AZD2858, AZD1080, SB415286, LY2090314, AR-A014418, SB216763, AR-A014418, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, and GF109203X.

In preferred embodiments, the Wnt signaling pathway activator is selected from Kenpaullone, 1-Azakenpaullone, CHIR99021, CHIR98014, NP031112, TWS119, AZD2858, AZD1080, SB415286, LY2090314, AR-A014418, CT 99021, CT 20026, SB216763, AR-A014418, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(1-pyridyl) [1,3,4]-oxadiazole, OTDZT, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, TWS1 19 pyrrolopyrimidine compound, L803 H-KEAPPAPPQSpP-NH2 or its myristoylated form, 2-Chloro-1-(4,5-dibromo-thiophen-2-yl)-ethanone, GF109203X, RO318220, TDZD-8, TIBPO, or OTDZT. In a most preferred embodiment, the Wnt signaling pathway activator is CHIR99021.

### Additional Reagents

In addition to basal medium, the compositions (e.g., the first to fifth differentiation culture media) of the present disclosure may have or comprise one or more additional reagents as supplement(s). Depending on the requirements of the present disclosure, one or more additional reagents may be independently added into the basal media of the first to fifth differentiation culture media before or upon use of the culture media. Additional reagents can include, for example, one or more cytokines (e.g., cytokine(s) that stimulate the hematopoietic differentiation). The concentrations of one or more cytokines in the medium are not particularly limited as long as they stimulate the differentiation of pluripotent stem cells into hematopoietic lineage cells including HE, HP or iNK cells.

### Cytokines

Cytokines are a group of cell signaling molecules including, for example, growth Factors, interleukins, colony stimulating factors, chemokines, interferons, lymphokines, and tumor necrosis factors. For example, examples of interleukins include, without limitation, IL-1, IL-2, IL-3, IL-6, IL-7, IL-9, IL-10, IL-12, IL-15, IL-17, IL-18, IL-21 and IL-27. For example, examples of colony stimulating factors include, without limitation, stem cell factor (SCF), Flt-3 ligand (Flt3L), thrombopoietin (TPO), G-CSF, and GM-CSF. For example, examples of tumor necrosis factors include, without limitation, tumor necrosis factor alpha (TNFα) and tumor necrosis factor beta (TNF-β). For example, examples of interferons include, without limitation, IFN-γ, IFN-κ, IFN-1, IFN-2, IFN-3, and IFN-4.

### Growth Factors

Growth factors are molecules capable of stimulating a variety of cellular processes, including, for example, cell proliferation, cell migration, differentiation, and multicellular morphogenesis during development and tissue healing. Examples of growth factors include, without limitation, Bone Morphogenetic Factors (BMPs), Epidermal Growth Factors (EGFs), Endothelial Cell Growth Factors (ECGFs), Fibroblast Growth Factors (FGFs), Insulin-like Growth Factors (IGFs), Nerve Growth Factors (NGFs), Platelet-derived Growth Factors (PDGFs), and Vascular Endothelial Growth Factors (VEGFs).

In preferred embodiments, the first to third differentiation culture media of the present disclosure are supplemented with one or more growth factors. In certain embodiments, the first and second differentiation culture media are each supplemented with BMP4, VEGF, and bFGF. In certain embodiments, the third and fourth differentiation culture media are each supplemented with VEGF and bFGF.

The concentration of growth factor in the medium is not particularly limited, and may be for example, 1 to 200 ng/ml.

In preferred embodiments, the first and second differentiation culture media of the present disclosure are supplemented with one or more BMP signaling pathway activators. Preferred examples of BMP signaling pathway activators include BMP2, BMP4, SB4, Ventromorphins (SJ000291942, SJ000063181, SJ000370178), Isoliquiritigenin, Diosmetin, Apigenin, Biochanin. A most preferred embodiment of the BMP signaling pathway activator used in the present disclosure is BMP4. The concentration of BMP4 in the medium is not particularly limited as long as it activates a BMP signaling pathway, and examples thereof include, but are not limited to 1 to 200 ng/ml, such as 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 dng/ml, 130 ng/ml, 140 ng/ml, 150 ng/ml, 160 ng/ml, 180 ng/ml, 200 ng/ml.

In preferred embodiments, the first to fourth differentiation culture media of the present disclosure are each supplemented with VEGF. In preferred embodiments, the first to fourth differentiation culture media are each independently further supplemented with a VEGF at a concentration of 15 to 100 ng/mL. In certain embodiments, the first to third differentiation culture media are each independently further supplemented with a VEGF at a concentration of 25 to 100 ng/mL, and preferably 50 to 100 ng/mL, because the higher concentrations of VEGF in the first to third differentiation culture media can facilitate the differentiation into HP and immature iNK cells.

In certain embodiments, the first to fourth differentiation culture media of the present disclosure are each supplemented with VEGF at a concentration of about 15, 20, 25, 30, 40, 50, 60, 70, 80, 90, or 100 ng/mL. In certain embodiments, the first to fourth differentiation culture media of the present disclosure are each supplemented with VEGF at a concentration of about 20-50 ng/mL.

In preferred embodiments, the first to fourth differentiation culture media of the present disclosure are each supplemented with bFGF. In preferred embodiments, the first to fourth differentiation culture media are each independently further supplemented with a bFGF at a concentration of 0.1 to 20 ng/mL.

In certain embodiments, the first to fourth differentiation culture media of the present disclosure are each supplemented with bFGF at a concentration of about 0.5 ng/ml. In certain embodiments, the first to fourth differentiation culture media of the present disclosure are each supplemented with bFGF at a concentration of about 5 ng/ml. In certain embodiments, the first to fourth differentiation culture media of the present disclosure each are each supplemented with bFGF at a concentration of about 10 ng/ml. In certain embodiments, the first to fourth differentiation culture media of the present disclosure are each supplemented with bFGF at a concentration of about 0.1-15 ng/ml. In certain embodiments, the first to fourth differentiation culture media of the present disclosure are each supplemented with bFGF at a concentration of about 1-10 ng/mL. In certain embodiments, the first to fourth differentiation culture media of the present disclosure are each supplemented with bFGF at a concentration of about 5-10 ng/ml.

### Colony Stimulating Factors

Colony stimulating factors are cytokines capable of stimulating hematopoietic stem or progenitor cell differentiation and proliferation. Examples of colony stimulating factors include, without limitation, stem cell factor (SCF), Flt-3 ligand (Flt3L), thrombopoietin (TPO), G-CSF, GM-CSF and multi-CSF.

In preferred embodiments, the fourth to fifth differentiation culture media of the present disclosure are each supplemented with one or more colony stimulating factors. The concentration of colony stimulating factor in the medium is not particularly limited as long as they stimulate the differentiation and proliferation of hematopoietic stem or progenitor cells, and may be for example, 1 to 200 ng/ml, and examples thereof include, but are not limited to, TPO at a concentration of 1 to 100 ng/ml, such as 5 ng/ml, 10 ng/ml, 15 ng/ml, 20 ng/ml, 30 ng/ml, 40 ng/ml, 50 ng/ml, 60 ng/ml, 70 ng/ml, 80 ng/ml, 90 ng/ml, 100 ng/ml; SCF at a concentrations of 1 to 200 ng/ml, such as 1 ng/ml, 10 ng/ml, 20 ng/ml, 30 ng/ml, 40 ng/ml, 50 ng/ml, 60 ng/ml, 80 ng/ml, 100 ng/ml, 150 ng/ml, 200 ng/ml; Flt-3L at a concentrations of 1 to 200 ng/ml such as 1 ng/ml, 5 ng/ml, 10 ng/ml, 20 ng/ml, 30 ng/ml, 40 ng/ml, 50 ng/ml, 80 ng/ml, 100 ng/ml, 150 ng/ml, 200 ng/ml.

In certain embodiments, the fourth differentiation culture medium is supplemented with SCF, Flt3L, TPO, VEGF and bFGF.

### Interleukins

Interleukins are cytokines capable of stimulating hematopoietic stem or progenitor cell differentiation and proliferation. Examples of interleukins include, without limitation, IL-1, IL-2, IL-3, IL-6, IL-7, IL-9, IL-10, IL-12, IL-15, IL-17, IL-18, IL-21 and IL-27. In preferred embodiments, the fifth differentiation culture medium of the present disclosure is supplemented with one or more interleukins. In a most preferred embodiment, the fifth differentiation culture medium is supplemented with IL-7, IL-3, IL-2, IL-15. The concentration of interleukin in the medium is not particularly limited, and may be for example, 1 to 200 ng/ml.

In certain embodiments, the fifth differentiation culture medium of the present disclosure is supplemented with IL-7 at a concentration of about 10 ng/ml. In certain embodiments, the fifth differentiation culture medium of the present disclosure is supplemented with IL-7 at a concentration of about 25 ng/mL. In certain embodiments, the fifth differentiation culture medium of the present disclosure is supplemented with IL-7 at a concentration of about 50 ng/mL. In certain embodiments, the fifth differentiation culture medium of the present disclosure is supplemented with IL-7 at a concentration of about 1-50 ng/mL.

In certain embodiments, the fifth differentiation culture medium of the present disclosure is supplemented with IL-3 at a concentration of about 5 ng/ml. In certain embodiments, the fifth differentiation culture medium of the present disclosure is supplemented with IL-3 at a concentration of about 10 ng/mL. In certain embodiments, the fifth differentiation culture medium of the present disclosure is supplemented with IL-3 at a concentration of about 20 ng/mL. In certain embodiments, the fifth differentiation culture medium of the present disclosure is supplemented with IL-3 at a concentration of about 1-20 ng/mL.

In certain embodiments, the fifth differentiation culture medium of the present disclosure is supplemented with IL-2 at a concentration of about 100 IU/mL. In certain embodiments, the fifth differentiation culture medium of the present disclosure is supplemented with IL-2 at a concentration of about 400 IU/mL. In certain embodiments, the fifth differentiation culture medium of the present disclosure is supplemented with IL-2 at a concentration of about 700 IU/mL. In certain embodiments, the fifth differentiation culture medium of the present disclosure is supplemented with IL-2 at a concentration of about 10-700 IU/mL.

In certain embodiments, the fifth differentiation culture medium of the present disclosure is supplemented with IL-15 at a concentration of about 10 ng/ml. In certain embodiments, the fifth differentiation culture medium of the present disclosure is supplemented with IL-15 at a concentration of about 20 ng/mL. In certain embodiments, the fifth differentiation culture medium of the present disclosure is supplemented with IL-15 at a concentration of about 50 ng/mL. In certain embodiments, the fifth differentiation culture medium of the present disclosure is supplemented with IL-15 at a concentration of about 1-50 ng/mL.

In certain embodiments, the fifth differentiation culture medium of the present disclosure is supplemented with IL-10 at a concentration of about 10 ng/ml. In certain embodiments, the fifth differentiation culture medium of the present disclosure is supplemented with IL-10 at a concentration of about 20 ng/ml. In certain embodiments, the fifth differentiation culture medium of the present disclosure is supplemented with IL-10 at a concentration of about 50 ng/mL. In certain embodiments, the fifth differentiation culture medium of the present disclosure is supplemented with IL-10 at a concentration of about 1-50 ng/mL.

In certain embodiments, the fifth differentiation culture medium of the present disclosure is supplemented with IL-18 at a concentration of about 20 ng/ml. In certain embodiments, the fifth differentiation culture medium of the present disclosure is supplemented with IL-18 at a concentration of about 50 ng/mL. In certain embodiments, the fifth differentiation culture medium of the present disclosure is supplemented with IL-18 at a concentration of about 100 ng/mL. In certain embodiments, the fifth differentiation culture medium of the present disclosure is supplemented with IL-18 at a concentration of about 20-100 ng/mL.

In certain embodiments, the fifth differentiation culture medium is supplemented with SCF, Flt3L, TPO, IL-7, IL-3, IL-2, and IL-15.

In certain embodiments, the first to fifth differentiation culture media are chemically defined serum-free and xeno-free differentiation culture media. According to the above embodiments, such media can avoid potential risk for xenogenic contamination, have reduced difference between batches, and can be better suitable for clinical and therapeutic use.

In preferred embodiments, the step of contacting the PSCs with a maintenance culture medium to form embryoid bodies (EBs) comprises forming the EBs from the PSCs by suspension maintenance culture, hanging drop EB formation or Spin EB formation. In more preferred embodiments, the EBs are formed by suspension maintenance culture.

In preferred embodiments, the maintenance medium contains a ROCK inhibitor. The maintenance medium can be for example, E8 or mTeSR or other similar medium. In more preferred embodiments, the ROCK inhibitor is selected from the group consisting of Y27632, Blebbistatin, HA100, HA1152, HA-1077, and any combination thereof. The concentration of ROCK inhibitor in the maintenance medium may be 1 to 20 μM such as 10 μM.

In preferred embodiments, the method of the present disclosure is carried out under 3D culture condition. According to the above embodiments, the 3D differentiation system greatly saves culture space and culture volume and is simpler and easier, and remarkably increases the number of cells obtained, thereby being beneficial to the large-scale production of the hematopoietic lineage cells such as HE cells, HP cells and iNK cells from hPSCs.

### Second Differentiation Method

The second aspect of the present disclosure relates to a method for promoting the directed differentiation (e.g., hematopoietic differentiation) of pluripotent stem cells (PSCs) (into e.g., hematopoietic lineage cells), comprising the steps of: contacting the PSCs with a maintenance culture medium to form embryoid bodies (EBs); contacting the EBs with a first differentiation culture medium supplemented with a Wnt signaling pathway activator, or with a first differentiation culture medium supplemented with a Wnt signaling pathway activator and a second differentiation culture medium supplemented with a Wnt signaling pathway activator sequentially, to form mesodermal cells; and contacting the mesodermal cells with a third differentiation culture medium supplemented with a Wnt signaling pathway inhibitor to obtain hemogenic endothelial (HE) cells. According to the second aspect of the present disclosure, due to the stage-specific use of a Wnt signaling pathway inhibitor in conjunction with a Wnt signaling pathway activator in the process of directed differentiation of pluripotent stem cells (PSCs), the differentiation efficiency for HE cells can be improved. The above method may be accomplished within about 3 days and produce a high number of HE cells.

In certain embodiments, the method of present disclosure further comprises the step of contacting the HE cells with a fourth differentiation culture medium to obtain hematopoietic progenitor (HP) cells. According to the above embodiments, due to the stage-specific use of a Wnt signaling pathway inhibitor in conjunction with a Wnt signaling pathway activator in the process of directed differentiation of pluripotent stem cells (PSCs) into HP cells, the differentiation efficiency for HP cells can be improved. The above method may be accomplished within about 6-12 days and produce a high number of HP cells. For example, 9×10⁶ HP cells can be derived from 1×10⁶ hPSCs.

The step of contacting the HE cells with a fourth differentiation culture medium to obtain hematopoietic progenitor (HP) cells can be conducted according to conventional method in the art, or any other suitable method, such as the corresponding method described elsewhere herein (e.g., as described in the first differentiation method or the first aspect herein), and these same descriptions are omitted herein for purpose of simplification.

In certain embodiments, the method of present disclosure further comprises the step of contacting the HP cells with a fifth differentiation culture medium to obtain immature iNK cells. According to the above embodiments, due to the stage-specific use of a Wnt signaling pathway inhibitor in conjunction with a Wnt signaling pathway activator in the process of directed differentiation of pluripotent stem cells (PSCs) into iNK cells, the differentiation efficiency for immature iNK cells can be improved.

The step of contacting the HP cells with a fifth differentiation culture medium to obtain immature iNK cells can be conducted according to conventional method in the art, or any other suitable method, such as the corresponding method described elsewhere herein (e.g., as described in the first differentiation method or the first aspect herein), and these same descriptions are omitted herein for purpose of simplification.

The step of contacting the PSCs with a maintenance culture medium to form embryoid bodies (EBs) can be conducted according to the corresponding method described elsewhere herein (e.g., as described in the first differentiation method or the first aspect herein), and these same descriptions are omitted herein for purpose of simplification.

In preferred embodiments, the EBs are contacted with the first differentiation culture medium and the second differentiation culture medium sequentially to form mesodermal cells, wherein the Wnt signaling pathway activator in the second differentiation culture medium may be same or different and has an equal or lower concentration as compared to the Wnt signaling pathway activator in the first differentiation culture medium. According to the above embodiments, by fine-tuning (firstly activating and then suppressing the Wnt signaling of cells, or down-regulating the Wnt signaling stepwise) the Wnt signaling during hematopoietic differentiation, the differentiation efficiency for HE cells, HP cells or immature NK cells can be further improved.

In preferred embodiments, the second differentiation culture medium has the same composition as that of the first differentiation culture medium except that the concentration of the Wnt signaling pathway activator in the second differentiation culture medium is lower than the concentration of the Wnt signaling pathway activator in the first differentiation culture medium. According to the above embodiments, due to the smoother transition from the activation to the suppression of Wnt signaling, the differentiation into HE cells, HP (CD34+) cells or iNK (CD56+) cells can be further facilitated.

Examples the Wnt signaling pathway activator and its concentrations in the first and second differentiation culture media have been described elsewhere herein (e.g., as described in the first differentiation method or the first aspect herein), and these same descriptions are omitted herein for purpose of simplification.

In preferred embodiments, the Wnt signaling pathway activator is selected from the group consisting of Kenpaullone, 1-Azakenpaullone, CHIR99021, CHIR98014, NP031112, TWS119, AZD2858, AZD1080, SB415286, LY2090314, AR-A014418, SB216763, AR-A014418, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, GF109203X, and any combination thereof. In a most preferred embodiment, the Wnt signaling pathway activator is CHIR99021.

In preferred embodiments, the concentration of the Wnt signaling pathway activator in the second differentiation culture medium is from 0 to 4 μM, and the concentration of the Wnt signaling pathway activator in the first differentiation culture medium is from 4 to 8 μM. In more preferred embodiments, the concentration of the Wnt signaling pathway activator in the second differentiation culture medium is from 0 to 4 μM, and the concentration of the Wnt signaling pathway activator in the first differentiation culture medium is from 5 to 8 μM.

Examples the Wnt signaling pathway inhibitor and its concentration in the third differentiation culture medium have been described elsewhere herein (e.g., as described in the first differentiation method or the first aspect herein).

In preferred embodiments, the Wnt signaling pathway inhibitor is selected from the group consisting of iCRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, Isoquercitrin, Gigantol, JW55, MSAB, IWR-1-endo, FH535, WIKI4, CCT251545, KYA1797K, NCB-0846, iCRT14, Adavivint, M435-1279, XAV939, and any combination thereof. In a most preferred embodiment, the Wnt signaling pathway inhibitor is XAV939.

In preferred embodiments, the concentration of the Wnt signaling pathway inhibitor in the third differentiation culture medium is from 1 to 30 μM.

In preferred embodiments, the third differentiation culture medium is further supplemented with a TGF-β signaling pathway inhibitor. According to the above embodiments, the differentiation efficiency for HE cells or HP (CD34+) cells cells can be further improved.

In certain embodiments, the third differentiation culture medium of the present disclosure is supplemented with a TGF-β signaling pathway inhibitor at a concentration of about 1-20 μM, 1-15 μM, 1-10 μM such as about 0.1 μM, 0.5 μM, 1 M, 2 μM, 3 μM, 4 μM, 5 μM, 6 μM, 7 μM, 8 μM, 9 μM, 10 μM, 15 μM, or 20 μM.

### TGF-β Signaling Pathway Inhibitor

Transforming Growth Factor β (TGF-β) is part of a larger superfamily of secreted dimeric multifunctional proteins that also includes, for example, activins and bone morphogenetic proteins. TGF-β plays important roles in a plurality of cellular functions including embryogenesis, maintenance of tissue homeostasis in multicellular organisms, tumor suppression.

TGF-β signaling pathway inhibitors (also referred to as “TGF-β inhibitors”) refers to antagonists of the TGF-β signaling pathway (e.g., agents capable of downregulating activity and/or amount of a component participating in the TGF-β signaling pathway), including, but not limited to an antagonist of one or more of TGF-β1, TGF-β2, TGF-β3, TGF-β receptors Type I (TβRI), TGF-β receptors Type II (TβRII), and TGF-β receptors Type III (TβRIII). Non-limiting examples of TβRI include, ALK1, ALK2, ALK3, ALK4, ALK5, ALK6, and ALK7. Non-limiting examples of TβRII include, TGFβR2, BMPR2, ACVR2A, ACVR2B, AMHR2. TβRIII includes, for example, TGFBR3.

Examples of TGF-β signaling pathway inhibitors also include, without limitation, RepSox (2-[5-(6-methylpyridin-2-yl)-1H-pyrazol-4-yl]-1,5-naphthyridine), A83-01, SB431542, D4476, GW788388, LY364947, LY580276, SB525334, SB505124, SD208, GW6604, and GW788388. In preferred embodiments, a TGF-β signaling pathway inhibitor comprises SB431542.

Examples of the basal media of the first to third differentiation culture media have been described elsewhere herein (e.g., as described in the first differentiation method or the first aspect herein), and these same descriptions are omitted herein for purpose of simplification.

In certain embodiments, the first to third differentiation basal media comprise the same basal medium. According to the above embodiments, the differentiation protocol of the present disclosure can be simplified.

In certain embodiments, the basal media of the first to third differentiation culture media may be IF-4 or CD34A basal medium, and preferably IF-4 basal medium.

In certain embodiments, the basal medium of the fourth differentiation culture medium may be IF-4 or CD34A basal medium.

In case where the method of present disclosure further comprises the step of contacting the HE cells with a fourth differentiation culture medium to obtain HP cells, the basal medium of the fourth differentiation culture medium may be a basal medium supplemented with (i) a nicotinamide-based compound, and (ii) a heparin-based compound, and preferably, the basal medium of the fourth differentiation culture medium may be a basal medium supplemented with the combination of (i) a nicotinamide-based compound, (ii) a heparin-based compound, and (iii) a human platelet lysate. According to the above embodiments, the differentiation efficiency for HP (CD34+) can be further improved.

The examples and concentrations of the nicotinamide-based compound, the heparin-based compound and the human platelet lysate in the basal medium of the fourth differentiation culture medium have been described elsewhere herein (e.g., as described in the first differentiation method or the first aspect herein), and these same descriptions are omitted herein for purpose of simplification.

In certain embodiments, the concentration of the nicotinamide-based compound in the fourth differentiation culture medium is from 0.5 to 20 mM.

In certain embodiments, the concentration of the heparin-based compound in the fourth differentiation culture medium is from 0.1 to 100 μg/mL.

In certain embodiments, the concentration of the human platelet lysate in the fourth differentiation culture medium is from 0.1% to 20% by volume.

In preferred embodiments, the nicotinamide-based compound comprises nicotinamide, and the heparin-based compound comprises heparin sodium.

In preferable embodiments, the basal medium of the fourth differentiation culture medium contains IF-4 basal medium in addition to the combination of (i) a nicotinamide-based compound, (ii) a heparin-based compound, and (iii) a human platelet lysate. In more preferable embodiments, the basal medium of the fourth differentiation culture medium contains IF-4 basal medium in addition to the combination of (i) nicotinamide, (ii) heparin sodium, and (iii) human platelet lysate. In a most preferable embodiment, the basal medium of the fourth differentiation culture medium comprises a NKSFM basal medium.

In case where the method of present disclosure further comprises the step of contacting the HP cells with a fifth differentiation culture medium to obtain immature iNK cells, the basal medium of the fifth differentiation culture medium may be IF-4 or CD34A basal medium, and preferably IF-4 basal medium.

### Additional Reagents

In addition to basal medium, compositions (e.g., the first to third differentiation culture media in case where HE cells are produced, the first to fourth differentiation culture media in case where HP cells are produced, and the first to fifth differentiation culture media in case where immature cells are produced) of the second aspect of the present disclosure may have or comprise one or more additional reagents as supplement(s). Depending on the requirements of the present disclosure, one or more additional reagents may be independently added into the basal media of the first to fifth differentiation culture media before or upon use of the culture media. Additional reagents can include, for example, one or more cytokines (e.g., cytokine(s) that stimulate the hematopoietic differentiation). The concentrations of one or more cytokines in the medium are not particularly limited as long as they stimulate the differentiation of pluripotent stem cells into hematopoietic lineage cells including HE, HP or iNK cells.

The examples and the concentrations of the corresponding additional reagents have been described elsewhere herein (e.g., as described in the first differentiation method or the first aspect herein), and these same descriptions are omitted herein for purpose of simplification.

In certain embodiments, the first to fourth differentiation culture media are each independently further supplemented with a VEGF at a concentration of 15 to 100 ng/ml. In certain embodiments, the first to third differentiation culture media are each independently further supplemented with a VEGF at a concentration of 25 to 100 ng/mL, and preferably 50 to 100 ng/mL, because the higher concentrations of VEGF in the first to third differentiation culture media can facilitate the differentiation into HP and immature iNK cells.

In certain embodiments, the first to fifth differentiation culture media are chemically defined serum-free and xeno-free differentiation culture media. According to the above embodiments, such media can avoid potential risk for xenogenic contamination, have reduced difference between batches, and can be better suitable for clinical and therapeutic use.

In preferred embodiments, the method is carried out under 3D culture condition. According to the above embodiments, the 3D differentiation system greatly saves culture space and culture volume and is simpler and easier, and remarkably increases the number of cells obtained, thereby being beneficial to the large-scale production of the hPSCs to the hematopoietic lineage cells such as HE cells, and later HP cells, NK cells or other hematopoietic lineage cells such as T cells.

### Culture Medium

The third aspect of the present disclosure relates to a culture medium for promoting the directed differentiation of pluripotent stem cells (PSCs) into hematopoietic lineage cells, comprising a basal medium and supplemented with a Wnt signaling pathway inhibitor. According to the third aspect of the present disclosure, by using a Wnt signaling pathway inhibitor in the process for promoting the directed differentiation of pluripotent stem cells (PSCs) into hematopoietic lineage cells, the above culture medium can improve the differentiation efficiency for HE (KDR+) cells, HP (CD34+) cells or immature iNK (CD56+) cells.

The basal medium used in the third aspect of the present disclosure may be a common basal medium in the art or any other basal medium as long as it does not impede the promotion of the directed differentiation of pluripotent stem cells (PSCs) into hematopoietic lineage cells. The above basal medium may be commercially available or may be formulated according to requirements, for example, by adding one or more additives into a common basal medium in the art. Examples of common basal medium in the art have been described elsewhere herein (e.g., as described in the first differentiation method or the first aspect herein), and these same descriptions are omitted herein for purpose of simplification. Examples of the above basal medium may comprise IF-4, and CD34A basal medium, for example.

The examples and the concentration of Wnt signaling pathway inhibitor have been described elsewhere herein (e.g., as described in the first and second aspects herein), and these same descriptions are omitted herein for purpose of simplification.

In preferred embodiments, the Wnt signaling pathway inhibitor is selected from the group consisting of iCRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, Isoquercitrin, Gigantol, JW55, MSAB, IWR-1-endo, FH535, WIKI4, CCT251545, KYA1797K, NCB-0846, iCRT14, Adavivint, M435-1279, XAV939, and any combination thereof. In a most preferred embodiment, the Wnt signaling pathway inhibitor comprises XAV939.

In preferred embodiments, the concentration of the Wnt signaling pathway inhibitor in the culture medium is from 1 to 30 μM.

In preferred embodiments, the culture medium is further supplemented with a TGF-β signaling pathway inhibitor. According to the above embodiments, the differentiation efficiency for HE cells or HP (CD34+) cells cells can be further improved.

The examples and the concentration of the TGF-β signaling pathway inhibitor have been described elsewhere herein (e.g., as described in the first and second aspects herein), and these same descriptions are omitted herein for purpose of simplification.

In preferred embodiments, the culture medium is further supplemented with one or more growth factors. The examples and the concentration of the growth factor have been described elsewhere herein (e.g., as described in the first and second aspects herein), and these same descriptions are omitted herein for purpose of simplification.

In preferred embodiments, the growth factor is selected from bFGF and/or VEGF. In a most preferred embodiment, the growth factor is selected from bFGF and VEGF.

In preferred embodiments, the culture medium is further supplemented with 15-100 ng/mL, and preferably 15-50 ng/ml of VEGF.

In preferred embodiments, the culture medium is a chemically defined serum-free and xeno-free differentiation culture medium.

### Kit

The fourth aspect of the present disclosure relates to a kit comprising the above culture medium (e.g., culture medium of the third aspect described herein).

In preferred embodiments, the kit further comprises a first differentiation culture medium supplemented with a Wnt signaling pathway activator.

The first differentiation culture medium and the Wnt signaling pathway activator have been described elsewhere herein (e.g., as described in the first and second aspects herein), and these same descriptions are omitted herein for purpose of simplification.

In preferred embodiments, the kit further comprises a second differentiation culture medium supplemented with a Wnt signaling pathway activator, wherein the Wnt signaling pathway activator in the second differentiation culture medium may be same or different and has an equal or lower concentration as compared to the Wnt signaling pathway activator in the first differentiation culture medium.

The second differentiation culture medium and the Wnt signaling pathway activator have been described elsewhere herein (e.g., as described in the first and second aspects herein), and these same descriptions are omitted herein for purpose of simplification.

In preferred embodiments, the second differentiation culture medium has the same composition as that of the first differentiation culture medium except that the concentration of the Wnt signaling pathway activator in the second differentiation culture medium is lower than the concentration of the Wnt signaling pathway activator in the first differentiation culture medium.

In preferred embodiments, the concentration of the Wnt signaling pathway activator in the second differentiation culture medium is from 0 to 4 μM, and the concentration of the Wnt signaling pathway activator in the first differentiation culture medium is from 4 to 8 μM. In more preferred embodiments, the concentration of the Wnt signaling pathway activator in the second differentiation culture medium is from 0 to 4 μM, and the concentration of the Wnt signaling pathway activator in the first differentiation culture medium is from 5 to 8 μM.

In preferred embodiments, the Wnt signaling pathway activators in the first and second differentiation culture media are each independently selected from the group consisting of Kenpaullone, 1-Azakenpaullone, CHIR99021, CHIR98014, NP031112, TWS119, AZD2858, AZD1080, SB415286, LY2090314, AR-A014418, SB216763, AR-A014418, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, GF109203X, and any combination thereof.

The first and second differentiation culture media may be each supplemented with one or more grow factors.

The examples and the concentration of the grow factor have been described elsewhere herein (e.g., as described in the first and second aspects herein), and these same descriptions are omitted herein for purpose of simplification.

In preferred embodiments, the first and second differentiation culture media are each supplemented with 15-100 ng/mL, and preferably 15-50 ng/ml of VEGF.

In preferred embodiments, all culture media in the kit are chemically defined serum-free and xeno-free differentiation culture media comprising the same basal medium.

### Method for Producing iNK Cells

The fifth aspect of the present disclosure relates to a method for producing iNK cells, comprising the above method for promoting the directed differentiation of pluripotent stem cells (PSCs) according to the first aspect, and the step for expanding and maturing the immature iNK cells (Stage 3 in FIG. 1). The above method may be accomplished within about 27-69 days and produce a high number of iNK cells. For example, greater than 3×10¹⁰ functional iNK cells can be derived from 1× 10⁶ hPSCs.

The above method according to the first aspect have been elsewhere herein (e.g., as described in the first differentiation method or the first aspect herein), and these same descriptions are omitted herein for purpose of simplification.

The step for expanding and maturing the immature iNK cells can be conducted by conventional method in the art or any other suitable method.

In preferred embodiments, the step for expanding and maturing the immature iNK cells can include contacting the immature iNK cells with an expansion and maturation culture medium comprising a basal medium supplemented with the combination of (i) a nicotinamide-based compound, (ii) a heparin-based compound, and (iii) a human platelet lysate in the presence or absence of feeder cells. According to the above embodiments, the NK cell expansion fold, the percentage of mature iNK cells (CD56+CD3-cells) and the cytotoxity toward cancer cells can be improved as compared with commercial kit.

For the basal medium of the expansion and maturation culture medium including the nicotinamide-based compound, heparin-based compound, and human platelet lysate, its description is same as the SFM basal medium described elsewhere herein (e.g., as described in the first aspect herein), and these same descriptions are omitted herein for purpose of simplification.

In preferred embodiments, the concentration of the nicotinamide-based compound in the expansion and maturation culture medium is from 0.5 to 20 mM.

In preferred embodiments, the concentration of the heparin-based compound in the expansion and maturation culture medium is from 0.1 to 100 μg/mL.

In preferred embodiments, the concentration of the human platelet lysate in the expansion and maturation culture medium is from 0.1% to 20% by volume.

In preferred embodiments, the expansion and maturation culture medium is further supplemented with one or more of IL-2, IL-10, IL-18, and SB431542. According to the above embodiments, the cytotoxity toward cancer cells by expanded and matured iNK cells can be improved. In more preferred embodiments, the expansion and maturation culture medium is further supplemented with IL-10, and/or IL-18, because NK cell expansion fold can be further improved. In a most preferred embodiment, the expansion and maturation culture medium comprises IL-18.

The concentration of interleukin such as IL-2, IL-10, IL-18 in the medium is not particularly limited.

In certain embodiments, the expansion and maturation culture medium of the present disclosure is supplemented with IL-2 at a concentration of about 10-700 IU/mL such as about 100 IU/mL.

In certain embodiments, the expansion and maturation culture medium of the present disclosure is supplemented with IL-10 at a concentration of about 5-100 ng/ml, and preferably 5-50 ng/ml such as 20 ng/ml.

In certain embodiments, the expansion and maturation culture medium of the present disclosure is supplemented with IL-18 at a concentration of about 5-100 ng/mL, and preferably 5-60 ng/ml such as 50 ng/mL.

In certain embodiments, the expansion and maturation culture medium of the present disclosure is supplemented with SB431542 at a concentration of about 1-20 μM.

In preferred embodiments, the nicotinamide-based compound comprises nicotinamide, and the heparin-based compound comprises heparin sodium.

In preferable embodiments, the basal medium of the expansion and maturation medium contains IF-4 basal medium in addition to the combination of (i) a nicotinamide-based compound, (ii) a heparin-based compound, and (iii) a human platelet lysate. In more preferable embodiments, the basal medium of the expansion and maturation medium contains IF-4 basal medium in addition to the combination of (i) nicotinamide, (ii) heparin sodium, and (iii) human platelet lysate. In a most preferable embodiment, the basal medium of the expansion and maturation medium comprises a NKSFM basal medium.

In certain embodiments, the step of expanding and maturing the immature iNK cells further comprises co-culturing the immature iNK cells with feeder cells. In some such embodiments, a plurality of rounds (e.g., 1, 2, 3, 4, 5, 6, 7, 8, 9, or 10 rounds) of feeder stimulations are utilized to expand and/or mature the iNK cells.

Optionally, immature iNK cells can be cryopreserved before the expansion and maturation. Cryopreserved iNK cells can be thawed later to continue the expansion and maturation process. Optionally, functional or mature iNK cells can be cryopreserved at the end of the step for expanding and maturing the immature iNK cells.

In preferred embodiments, the cryopreservation medium comprises a basal medium supplemented with Human Serum Albumin (HSA, 20-40 mg/mL) and DMSO (5-10%, v/v). In more preferred embodiments, the basal medium is Multiple Electrolytes injection or Dextran injection.

In preferred embodiments, the expansion and maturation culture medium is a chemically defined serum-free and xeno-free culture media.

### Cell Populations

The present disclosure provides hematopoietic lineage cells or cell populations thereof, including HE cells, Hematopoietic Progenitor (HP) cells, immature Natural Killer (NK) cells, and mature NK cells and compositions comprising the same (“cell compositions”).

The present disclosure provides a cell population, wherein at least 50% of the cells in the population without any enrichment or purification are KDR+HE cells. In preferred embodiments, at least 60% of the cells in the population without any enrichment or purification are KDR+HE cells. In more preferred embodiments, at least 70% of the cells in the population without any enrichment or purification are KDR+HE cells. In most preferred embodiments, at least 80% of the cells in the population without any enrichment or purification are KDR+HE cells. The above cell population with high proportion of HE cells is a relatively homogeneous population, has better differentiation potential, and can be used for later differentiation (such as HP differentiation) without any purification or enrichment. The above cell population may be produced by the methods elsewhere herein (e.g. the methods described in the first and/or second differentiation methods herein).

The present disclosure also provides a cell population, wherein at least 40% of the cells in the population without any enrichment or purification are CD34+CD43− HP cells. In preferred embodiments, at least 50% of the cells in the population without any enrichment or purification are CD34+CD43− HP cells. In more preferred embodiments, at least 60% of the cells in the population without any enrichment or purification are CD34+CD43− HP cells. In further preferred embodiments, at least 70% of the cells in the population without any enrichment or purification are CD34+CD43− HP cells. In most preferred embodiments, at least 80% of the cells in the population without any enrichment or purification are CD34+CD43− HP cells. In certain embodiments, at least 40% of the cells in the population without any enrichment or purification are CD34+CD43−CD73− HP cells. The above cell population with high proportion of HP cells is a relatively homogeneous population, has better differentiation potential, and can be used for later differentiation (such as NK differentiation) without any purification or enrichment. The above cell population may be produced by the methods elsewhere herein (e.g. the methods described in the first and/or second differentiation methods herein).

The present disclosure further provides a cell population, wherein at least 40% of the cells in the population without any enrichment or purification are immature CD56+CD3− iNK cells. In certain embodiments, at least 50% of the cells in the population without any enrichment or purification are immature CD56+CD3− iNK cells. In further embodiments, at least 60% of the cells in the population without any enrichment or purification are immature CD56+CD3− iNK cells. In preferred embodiments, at least 70% of the cells in the population without any enrichment or purification are immature CD56+CD3− iNK cells. In more preferred embodiments, at least 80% of the cells in the population without any enrichment or purification are immature CD56+CD3− iNK cells. The above cell population with high proportion of iNK cells is a homogeneous population, has better expansion potential, and can be used for later expansion and maturation without any purification or enrichment. The above cell population may be produced by the methods elsewhere herein (e.g. the methods described in the first and/or second differentiation methods herein).

In particular, the sixth aspect of the present disclosure provides a cell population produced by the methods described in the first differentiation method or the first aspect described herein, or the methods described in the second differentiation method or the second aspect described herein.

In particular, the sixth aspect of the present disclosure also provides a cell population produced by the methods described in the method for producing iNK cells or the fifth aspect described herein.

In particular, the seventh aspect of the present disclosure further provides a cell population, wherein more than 90% of the cells in the population without any enrichment or purification are mature CD56+CD3− iNK cells. In preferred embodiments, at least 95% of the cells in the population without any enrichment or purification are mature CD56+CD3− iNK cells. In more preferred embodiments, at least 98% of the cells in the population without any enrichment or purification are mature CD56+CD3− iNK cells. In further preferred embodiments, at least 99% of the cells in the population without any enrichment or purification are mature CD56+CD3− iNK cells. In still further preferred embodiments, at least 99.5% of the cells in the population without any enrichment or purification are mature CD56+CD3− iNK cells. In most preferred embodiments, at least 99.8% (such as 99.9%, and 100%) of the cells in the population without any enrichment or purification are mature CD56+CD3− iNK cells. The above cell population with high proportion of functional or mature iNK cells is a highly homogeneous population (highly pure), has improved function (e.g. higher cytotoxicity towards tumor cells, improved homing to tissues and targets, secretion of high level of pro-inflammatory cytokines), and can be better applicable for clinical and therapeutic use without any purification or enrichment. The above cell population may be produced by the methods elsewhere herein (e.g. the methods described in the method for producing iNK cells or the fifth aspect described herein).

The functional or mature iNK cells and cell population thereof of the present disclosure have improved expression pattern of surface receptors compared to primary NK cells (e.g., higher expression of chemokine receptor such as CCR6 and lower expression of inhibitory receptors such as NKG2A and KIRe1, as compared to primary NK cells) or iNK cells reported in the literatures, which indicates better function.

The functional or mature iNK cells and cell population thereof of the present disclosure have lower expression of inhibitory receptors (e.g. NKG2A and KIRe1) as compared to primary NK cells, which indicates better function (e.g., activation toward target cells). In certain embodiments, the functional or mature iNK cells and cell population thereof of the present disclosure have at least 2 times lower expression of inhibitory receptors (e.g. NKG2A and KIRe1) as compared to primary NK cells, which indicates greatly improved function (e.g., activation toward target cells). In certain embodiments, the functional or mature iNK cells and cell population thereof of the present disclosure have at least 3 times lower expression of inhibitory receptors (e.g. NKG2A and KIRe1) as compared to primary NK cells.

As an example, less than 20% of the cells in the iNK cells are NKG2A+ cells. In preferred embodiments, less than 17% of the cells in the iNK cells are NKG2A+ cells.

As an example, less than 20% of the cells in the iNK cells are KIRe1+ cells. In preferred embodiments, less than 12% of the cells in the iNK cells are KIRe1+ cells.

The functional or mature iNK cells and cell population thereof of the present disclosure have higher expression of chemokine receptor as compared to primary NK cells, indicating better function (e.g. homing to tissues and targets) than primary NK cells.

In certain embodiments, the functional or mature iNK cells and cell population thereof of the present disclosure have at least 19 times higher expression of chemokine receptor (e.g. CCR6) as compared to primary NK cells, which indicates greatly improved function (e.g. homing to tissues and targets). In certain embodiments, the functional or mature iNK cells and cell population thereof of the present disclosure have at least 20 times higher expression of chemokine receptor (e.g. CCR6) as compared to primary NK cells.

As an example, at least 70% of the cells in the iNK cells are CCR6+ cells. In preferred embodiments, at least 80% of the cells in the iNK cells are CCR6+ cells.

In preferred embodiments, the functional or mature iNK cells and cell population thereof of the present disclosure have comparable expression of activating receptors such as NKG2D and NKp30 as compared with primary NK cells.

As an example, at least 60% of the cells in the iNK cells are NKG2D+ cells. In preferred embodiments, at least 70% of the cells in the iNK cells are NKG2D+ cells.

As an example, at least 80% of the cells in the iNK cells are NKp30+ cells. In preferred embodiments, at least 90% of the cells in the iNK cells are NKp30+ cells.

In certain embodiments, more than 90% of the cells in the iNK cells are CD45+ INK cells. In more preferred embodiments, more than 95% of the cells in the iNK cells are CD45+ iNK cells. In most preferred embodiments, more than 99%, including 99.1%, 99.2%, 99.3%, 99.4%, 99.5%, 99.6%, 99.7%, 99.8%, 99.9%, and 100%, of the cells in the iNK cells are CD45+ iNK cells.

In certain embodiments, less than 20%, including 19%, 18%, 17%, 16%, 15%, 14%, 13%, 12%, 11%, 10%, 9%, 8%, 7%, 6%, 5%, 4%, 3%, 2%, 1%, and 0%, of the iNK cells are NKG2A+. In certain embodiments, less than 20%, including 19%, 18%, 17%, 16%, 15%, 14%, 13%, 12%, 11%, 10%, 9%, 8%, 7%, 6%, 5%, 4%, 3%, 2%, 1%, and 0%, of the iNK cells are KIRe1+. In certain embodiments, at least 70%, including 70%, 75%, 80%, 85%, 90%, 95%, 99%, or 100%, of the iNK cells are CCR6+.

### Pharmaceutical Composition

The present disclosure provides a pharmaceutical composition comprising any of the cell populations as described in the Cell Populations described herein and a pharmaceutically acceptable carrier. The cells in the cell population can be in an effective amount suitable for use in the therapy of the condition or disease of a subject.

In particular, the eighth aspect of the present disclosure provides a pharmaceutical composition comprising the cell population as described in the seventh aspect of the present disclosure and a pharmaceutically acceptable carrier. The cells in the cell population can be in an effective amount suitable for use in the therapy of the condition or disease of a subject.

In certain embodiments, a cell population may be prepared as a pharmaceutical composition (e.g., comprising a pharmaceutically acceptable carrier or excipient). A cell population can also be emulsified or presented as a liposome composition, provided that the emulsification procedure does not adversely affect cell viability. The cells, or the cells and any other active ingredient can be mixed with excipients that are pharmaceutically acceptable and compatible with the active ingredient, and in effective amounts suitable for use in the therapeutic methods described herein.

Pharmaceutically acceptable carriers are well known in the art. Exemplary pharmaceutically acceptable carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of cell population used in the pharmaceutical composition that is effective in the treatment of a particular disorder or condition can depend on the nature of the disorder or condition and can be determined by standard clinical techniques.

Modes of administration include but are not limited to injection and infusion. In certain embodiments, injection includes, without limitation, intravenous, intrathecal, intraperitoneal, intraspinal, intracerebrospinal, and intrasternal infusion. In certain embodiments, the route is intravenous. In certain embodiments, cells described herein are administered as a bolus or by continuous infusion (e.g., intravenous infusion) over a period of time. In certain embodiments, cells described herein are administered in several doses over a period of time (e.g., several infusions over a period of time). The cells described herein can be administered in a single dose or in 2, 3, 4, 5, 6 or more doses (or infusions).

In certain embodiments, a pharmaceutical composition comprises a cell population that are allogeneic to a subject. In certain embodiments, a pharmaceutical composition comprises a cell population described herein that are autologous to a subject.

### Use

The hematopoietic lineage cells or cell populations thereof of the present disclosure can be widely used for the treatment or prevention of a variety of disorders or diseases, including cancers, autoimmune diseases and blood diseases. In particular, the cell population of the seventh aspect of the present disclosure have improved function (e.g. higher cytotoxicity towards tumor cells, improved homing to tissues and targets, secretion of high level of pro-inflammatory cytokines) and thus can be better applicable for clinical and therapeutic use in a cancer. The cell population of the seventh aspect of the present disclosure can also be used to kill a variety of microorganisms, such as virus, bacteria etc, and old cells etc.

The present disclosure also provides a use of any of the cell populations described herein in the manufacture of a medicament for treating or preventing a cancer, an autoimmune disease or a blood disease.

In particular, the ninth aspect of the present disclosure provides a use of the cell population of the seventh aspect in the manufacture of a medicament for treating or preventing a cancer.

A wide range of cancers can be treated or prevented by administration of a cell population or pharmaceutical composition of the disclosure to a subject in need thereof.

Examples of cancers include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, mesothelioma, myelomas, nasopharynx cancers, neuroblastomas, non-Hodgkin's lymphoma, oral cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, chronic myelogenous leukemia, acute myeloid leukemia, myelomonocytic leukemia, melanoma, large cell membrane lung cancer, ovarian cancer, non-small-cell lung cancer, or small-cell lung cancer, and metastases thereof. In preferred embodiments, the cancer comprises chronic myelogenous leukemia, acute myeloid leukemia, myelomonocytic leukemia, melanoma, large cell membrane lung cancer, ovarian cancer, non-small-cell lung cancer, or small-cell lung cancer. In more preferred embodiments, the cancer is acute myeloid leukemia, melanoma, small-cell lung cancer, large cell membrane lung cancer, ovarian cancer, or non-small-cell lung cancer. The cell population or pharmaceutical composition of the disclosure has much better effects for treating or preventing these cancers as compared to primary NK cells.

In an additional aspect, the present disclosure provides methods of treating a subject in need thereof by administering to the subject a cell population (e.g., according to the seventh aspect) or pharmaceutical composition (e.g., according to the eight aspect) as described herein. Pharmaceutical compositions, cell compositions or populations of the present disclosure can be administered before, during, and/or after the onset of a disease, disorder, and/or condition (e.g., cancer).

In certain embodiments, a subject has a disease, disorder, or condition that can be treated by a cell-based therapy. In certain embodiments, a subject in need of a cell-based therapy is a subject with a disease, disorder and/or condition, whereby a cell-based therapy, e.g., a therapy in which a pharmaceutical composition or cell population described herein is administered to the subject, whereby the cell-based therapy treats at least one symptom associated with the disease, disorder, and/or condition.

### Method for Characterization

Methods of characterizing hematopoietic lineage cells including HE cells, HP cells and NK cells (e.g., iNK cells described herein), including characterization of cellular phenotype and/or functionality are known to those of skill in the art. Such methods include, but are not limited to, morphological analyses, flow cytometry, and/or gene expression profiling. One or more cell markers may be determined using one or more characterization methods to determine a composition, phenotype, and/or functionality of one or more cells and/or cell populations produced by the compositions and/or methods described herein. For example, in certain embodiments, cells of a particular population are characterized using gene expression profiling. In some such embodiments, a sample of a population of cells or a cell composition is evaluated for transcriptional signatures characteristic of a particular cell type (e.g., primary NK cells).

In an additional example, cells of a particular population or a cell composition can be characterized by flow cytometry. In certain embodiments, a sample of a population of cells can be evaluated for presence and/or proportion of one or more cell surface markers and/or one or more intracellular markers. As will be understood by those of skill in the art, cell surface markers may be representative of different lineages. For example, pluripotent cells may be identified by one or more of any number of markers to be associated with such cells, including, for example, CD34. Cells (e.g., cell compositions described herein) can be identified by markers that indicate some degree of differentiation (e.g., partial differentiation). For example, markers of differentiated cells can include those associated with hematopoietic progenitor cells such as, for example, CD45, CD34. In certain embodiments, markers of differentiated cells may be associated with NK cells, such as CD56, CD3, CD45, natural killer group-2 member A (NKG2A), killer immunoglobulin-like receptor (KIR) (e.g., KIRe1), C-C Motif Chemokine Receptor 6 (CCR6), NKG2D and NKp30.

### General Methods

In practicing the present disclosure, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### WORKING EXAMPLES

### Materials and Methods

All reagents for the culture media and apparatuses utilized throughout the Examples of the present disclosure are commercially available. The steps of the methods utilized throughout the Examples will be described with reference to FIG. 1.

### Examples 1-4: Development of NKSFM

The present examples demonstrate the process of developing NKSFM, an exemplary serum-free and xeno-free SFM basal medium. The development originated from the exploration of nicotinamide (NAM), Heparin sodium, Human Platelet Lysate in the expansion and maturation of PBNK cells as surrogate cells.

### Example 1

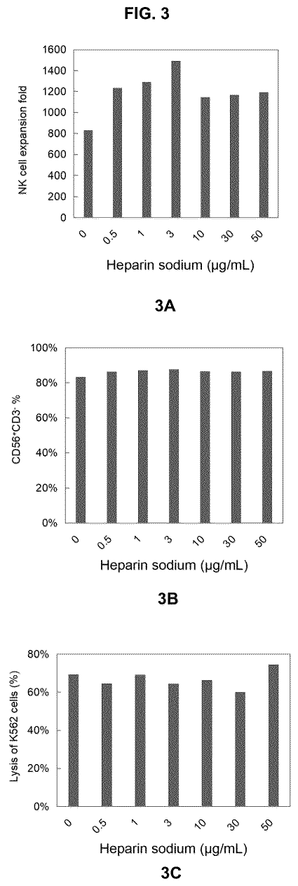

The effect of the concentration of nicotinamide (NAM) in an exemplary basal medium on NK cell expansion was evaluated. The above basal medium comprised the following components: IMDM (Sigma):RPMI1640 (Gibco) (50%: 50%); about 1.3 ng/ml of Cupric sulfate, about 3 μM of Ferric sulfate, about 0.432 μg/mL of Zinc sulfate, about 1˜50 ng/ml of Na Selenite, about 110 μg/mL of Sodium pyruvate, 0.1˜20 μg/mL of Insulin, 1˜200 μg/mL of Transferrin, about 1% (v/v) of GlutaMAX-1, 0.1˜20 mg/mL of HSA, 1˜400 μM of MTG, about 80 μg/mL of Ascorbic acid and indicated concentration of NAM. Peripheral blood mononuclear cells (PBMCs) were isolated by density gradient centrifugation using Ficoll-Paque. On Day 0, PBMCs were plated at 1×10⁶ cells/mL in a 48-well-plate in the basal medium with NAM (either 0, 1, 2, 2.5, 3, 3.5, 4, or 5 mM) and stimulated with the following reagents for 3 days: immobilized CD16 antibody (Biolegend, 302014) (135 ng/cm²), IL-2 (700 IU/mL), IL-15 (10 ng/ml) and OK432 (0.01 KE/mL). On day 3, cells were re-suspended in the above basal medium supplemented with IL-2 (700 IU/mL) and IL-15 (10 ng/mL). Fresh medium was added every other day to maintain cell density below 2×10⁶ cells/mL. Cells were collect on Day 14 and stained with CD56-PE and CD3-PerCP-Cy5.5 antibodies, the percentage of CD56⁺CD3⁻ NK cells was analyzed by flow cytometry. Results of FIG. 2 suggest that the concentration of NAM in the basal medium significantly impacts the NK cell expansion fold (FIG. 2A), while show minimal effects on the percentage of CD56⁺CD3⁻ NK cells (FIG. 2B).

### Example 2

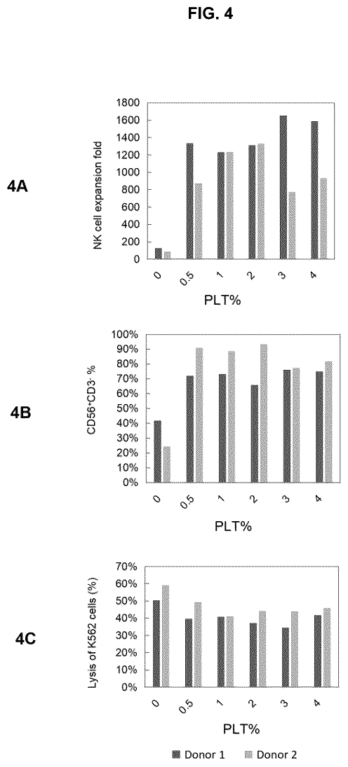

The effect of heparin sodium concentration in an exemplary NKSFM on NK cell expansion was also evaluated. NKSFM comprised the following components: IMDM (Sigma):DMEM/F12 (Gibco) (50%: 50%); about 20 μM of Ethanolamine, about 1˜50 ng/ml of Na Selenite, about 110 μg/mL of Sodium pyruvate, 0.1˜20 μg/mL of Insulin, 1˜200 μg/mL of Transferrin, about 1% (v/v) of GlutaMAX-1, 0.1˜20 mg/mL of Human serum albumin, 1˜400 μM of MTG, about 80 μg/mL of Ascorbic acid, about 1˜5 mM of NAM, 4% (v/v) of PLT, and indicated concentration of heparin sodium. PBMCs were isolated by density gradient centrifugation using Ficoll-Paque. On Day 0, PBMCs were plated at 1×10⁶ cells/mL in a 48-well-plate in NKSFM with heparin sodium (0, 0.5, 1, 3, 10, 30, or 50 μg/mL) and stimulated with the following reagents for 3 days: immobilized CD16 antibody (Biolegend, 302014) (135 ng/cm²), OK432 (0.01 KE/mL), NeoIL-2 (50 ng/ml), IL-12 (10 ng/mL), IL-18 (50 ng/mL). On day 3, cells were re-suspended in NKSFM supplemented with NeoIL-2 (10 ng/mL). Fresh medium was added every other day to maintain cell density below 2×10⁶ cells/mL. Cells were collect on Day 13 and stained with CD56-PE and CD3-PerCP-Cy5.5 antibodies, the percentage of CD56⁺CD3⁻ NK cells was analyzed by flow cytometry. The specific lysis of K562 tumor cells by NK cells were assessed by CFSE/7-AAD cytotoxicity assay. Briefly, K562 tumor cells were labelled with CFSE prior to co-culture with NK cells at E:T of 3:1. After incubation for 4 hours, cells were stained with 7-AAD (Live/dead staining) and analyzed with flow cytometry. Results of FIG. 3 indicate that the concentration of heparin sodium in NKSFM significantly influences the NK cell expansion fold (FIG. 3A), while show minimal effect on the percentage of CD56⁺CD3⁻ NK cells (FIG. 3B) and specific lysis of K562 tumor cells (FIG. 3C).

### Example 3

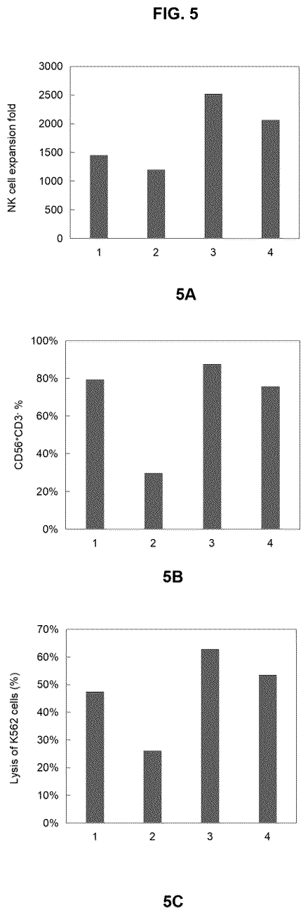

The effect of PLT percentage (v/v) in an exemplary NKSFM on NK cell expansion was evaluated. NKSFM comprised the following components: IMDM (Sigma):DMEM/F12 (Gibco) (50%: 50%); about 20 μM of Ethanolamine, about 1˜50 ng/ml of Na Selenite, about 110 μg/mL of Sodium pyruvate, 0.1˜20 μg/mL of Insulin, 1˜200 μg/mL of Transferrin, about 1% (v/v) of GlutaMAX-1, 0.1˜20 mg/mL of Human serum albumin, 1˜400 μM of MTG, about 80 μg/mL of Ascorbic acid, about 0.5-50 g/mL of heparin sodium, about 1˜5 mM of NAM, and indicated concentration of PLT. PBMCs were isolated by density gradient centrifugation using Ficoll-Paque. On Day 0, PBMCs were plated at 1×10⁶ cells/mL in a 48-well-plate in NKSFM with PLT (0, 0.5, 1, 2, 3, or 4%), and stimulated with the following reagents for 3 days: immobilized CD16 antibody (135 ng/cm²), OK432 (0.01 KE/mL), IL-12 (10 ng/mL), IL-15 (50 ng/mL), IL-18 (50 ng/mL). On day 3, cells were re-suspended in the basal medium supplemented with IL-2 (700 IU/mL) and IL-15 (10 ng/mL). Fresh medium was added every other day to maintain cell density below 2×10⁶ cells/mL. Cells were collect on Day 14 and stained with CD56-PE and CD3-PerCP-Cy5.5 antibodies, the percentage of CD56⁺CD3⁻ NK cells was analyzed by flow cytometry. The specific lysis of K562 tumor cells by NK cells were assessed by CFSE/7-AAD cytotoxicity assay. Briefly, K562 tumor cells were labelled with CFSE prior to co-culture with NK cells at E:T of 1:1. After incubation for 4 hours, cells were stained with 7-AAD (Live/dead staining) and analyzed with flow cytometry. As shown in FIG. 4, the concentration of PLT in NKSFM influences the expansion of NK cells at a certain degree (FIG. 4A), while show minimal effect on the percentage of CD56⁺CD3⁻ NK cells (FIG. 4B) and specific lysis of K562 tumor cells (FIG. 4C).

### Example 4

Lastly, an exemplary NKSFM was compared to commercial feeder-free or feeder-dependent NK cell expansion systems. The NKSFM comprised the following components: IMDM (Sigma):DMEM/F12 (Gibco) (50%: 50%); about 20 M of Ethanolamine, about 1˜50 ng/mL of Na Selenite, about 110 μg/mL of Sodium pyruvate, 0.1˜20 μg/mL of Insulin, 1˜200 μg/mL of Transferrin, about 1% (v/v) of GlutaMAX-1, 0.1˜20 mg/mL of Human serum albumin, 1˜400 μM of MTG, about 80 μg/mL of Ascorbic acid, about 1˜5 mM of NAM, 1% (v/v) of PLT and 0.5-50 μg/mL of heparin sodium. Four conditions were tested.

### Condition 1. NKSFM (Feeder-Free)

PBMCs were isolated by density gradient centrifugation using Ficoll-Paque. On Day 0, PBMCs were plated at 1×10⁶ cells/mL in 6-well-plates in NKSFM and stimulated with the following reagents for 3 days: immobilized CD16 antibody (Biolegend, 302014) (135 ng/cm²), OK432 (0.01 KE/mL), NeoIL-2 (50 ng/ml), IL-12 (10 ng/mL), IL-18 (50 ng/ml). On day 3, cells were re-suspended in NKSFM supplemented with NeoIL-2 (10 ng/mL). Fresh medium was added every other day to maintain cell density below 2×10⁶ cells/mL. Cells were collect on Day 14 and stained with CD56-PE and CD3-PerCP-Cy5.5 antibodies, the percentage of CD56⁺CD3⁻ NK cells was analyzed by flow cytometry. The specific lysis of K562 tumor cells by NK cells were assessed by CFSE/7-AAD cytotoxicity assay. Briefly, K562 tumor cells were labelled with CFSE prior to co-culture with NK cells at E:T of 3:1. After incubation for the indicated time, cells were stained with 7-AAD (Live/dead staining) and analyzed with flow cytometry.

### Condition 2. Commercial Kit (Feeder-Free)

A feeder-free commercial kit (Baso, 3.0A) was utilized as Condition 2. The feeder-free expansion of PBNK cells was conducted following the manufacturer's protocol. 5% (v/v) of autoplasma was added throughout the expansion.

Condition 3. NKSFM (with Feeder)

Feeder-dependent NKSFM was utilized as Condition 3. The feeder-dependent expansion of PBNK cells was conducted following the manufacturer's protocol except the plasma-containing medium was changed to NKSFM.

Condition 4. Commercial Kit (with Feeder)

Lastly, a feeder-dependent commercial kit (The Life ARK, ZY-NKZ-0104) was utilized as Condition 4. The feeder-dependent expansion of PBNK cells was conducted following the manufacturer's protocol (The Life ARK, CN). 10% (v/v) of autoplasma was added through the expansion.

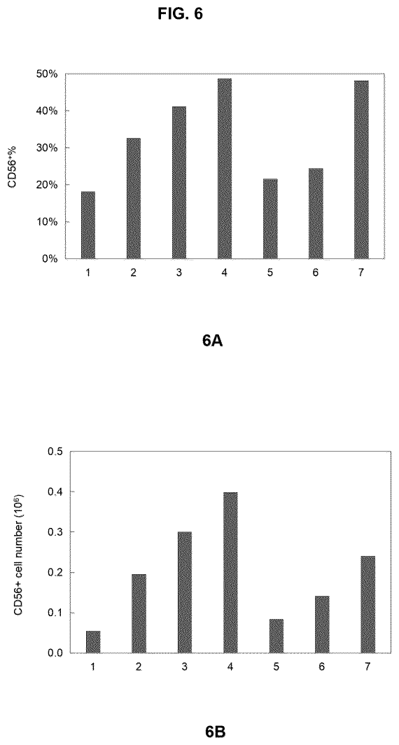

The results of FIG. 5 show that, in terms of NK cell expansion fold (FIG. 5A), the percentage of CD56+CD3− cells (FIG. 5B) and the lysis of K562 cells (FIG. 5C), NKSFM significantly outperforms the commercial kit in the absence of feeder cells, and also results in improved NK cell expansion compared to the commercial kit in the presence of feeder cells.

Taken together, the above experiments demonstrate that NKSFM is useful in the production of NK cells (e.g., for clinical use). NKSFM key components, NAM, heparin sodium, and PLT were found to be crucial for the expansion and function of NK cells. Most commercial NK cell expansion kits require serum and/or plasma to support NK cell expansion, or require sorting out the CD56⁺CD3⁻ NK cells before stimulation to avoid un-wanted expansion of CD3⁺ T cells in the PBMCs. However, use of NKSFM does not require either serum or plasma or sorting for preferential expansion of NK cells. Additionally, NKSFM can be used in both a feeder-free and feeder-dependent expansion of NK cells, both of which significantly outperformed commercial kits in both cell number and cytolytic activity. NKSFM can be not only used for expanding primary NK cells, but also iNK cells. Additionally, the present inventors further found that the above NKSFM can be also used in the process of directed differentiation of human pluripotent stem cells (hPSCs).

Exemplary basal media utilized throughout the Examples of the present disclosure and their components are summarized in Table 1.1.

TABLE 1.1
Components of exemplary basal media.

| # | Components | IF-4 | NKSFM | NKM | NKSFM-EP | CD34A |
|---|---|---|---|---|---|---|
| 1 | IMDM (Sigma) | + | + | + | + | + |
| 2 | DMEM/F12 (Gibco) | + | + | | | |
| 3 | GlutaMAX-1 (Invitrogen) | | + | | + | + |
| 4 | NEAA (Invitrogen) | | | + | | + |
| 5 | Bovine serum albumin | | | | | + |
| | (BSA, Sigma) | | | | | |
| 6 | Human serum albumin | + | + | + | + | |
| | (HSA, Sinopharm, CN) | | | | | |
| 7 | Monothioglycerol (MTG) | + | + | | | + |
| 8 | Ascorbic acid (Sigma) | + | + | | + | + |
| 9 | Transferrin (Sigma) | + | + | | + | + |
| 10 | Ferric sulfate (Sigma) | | | | + | |
| 11 | Ferric Nitrate (Sigma) | | | | + | |
| 12 | Na Selenite (Sigma) | + | + | | | |
| 13 | Trace elements A | | | | | + |
| | (Corning) | | | | | |
| 14 | Trace elements B | | | | | + |
| | (Corning) | | | | | |
| 15 | Trace elements C | | | | | + |
| | (Corning) | | | | | |
| 16 | Ethanolamine (Sigma) | + | + | | | |
| 17 | CD Lipid Concentrate | | | | + | + |
| | (Invitrogen) | | | | | |
| 18 | Sodium pyruvate (Sigma) | | + | | | |
| 19 | Insulin (Baiying, CN) | + | + | | + | |
| 20 | Nicotinamide (Sigma) | | + | + | + | |
| 21 | Heparin sodium | | + | | | |
| | (Thermo) | | | | | |
| 22 | Human Platelet Lysate | | + | | + | |
| | (PLT, BI) | | | | | |
| 23 | Mercaptoethanol | | | + | | |
| | (β-ME, Sigma) | | | | | |
| 24 | Human serum | | | + | | |

+ represents present, − represents absence, and +− represents present or absence.

Composition of IF-4: IMDM (Sigma):DMEM/F12 (Gibco) (50%: 50%); 0.1˜20 mg/mL of Human serum albumin (HSA, Sinopharm, CN); 1˜400 μM of Monothioglycerol (MTG); about 80 μg/mL of Ascorbic acid (Sigma); 1˜200 μg/mL of Transferrin (Sigma); 1˜50 ng/mL of Na Selenite (Sigma); about 20 μM of Ethanolamine (Sigma); and 0.1˜20 μg/mL of Insulin (Baiying, CN).

Composition of NKSFM: IMDM (Sigma):DMEM/F12 (Gibco) (50%: 50%); about 1% (v/v) of GlutaMAX-1 (Invitrogen); 0.1˜20 mg/mL of Human serum albumin (HSA, Sinopharm, CN); 1˜400 μM of Monothioglycerol (MTG); about 80 μg/mL of Ascorbic acid (Sigma); 1˜200 μg/mL of Transferrin (Sigma); about 1˜50 ng/ml of Na Selenite (Sigma); about 20 μM of Ethanolamine (Sigma); about 110 μg/mL of Sodium pyruvate (Sigma); 0.1˜20 μg/mL of Insulin (Baiying, CN); 1-5 mM of Nicotinamide (NAM, Sigma); 0.5˜50 μg/mL of Heparin sodium (Thermo); and 0.5˜4% (v/v) of Human Platelet Lysate (PLT, BI).

Composition of NKM: IMDM (Sigma) (100%); about 1% (v/v) of NEAA (Invitrogen); 0.1˜20 mg/mL of Human serum albumin (HSA, Sinopharm, CN); 1-5 mM of Nicotinamide (Sigma); about 50 M of Mercaptoethanol (β-ME, Sigma); and 10% (v/v) of Human serum.

Composition of NKSFM-EP: IMDM (Sigma) (100%); about 1% (v/v) of GlutaMAX-1 (Invitrogen); 0.1˜20 mg/mL of Human serum albumin (HSA, Sinopharm, CN); about 80 μg/mL of Ascorbic acid (Sigma); 1˜200 μg/mL of Transferrin (Sigma); about 3u M of Ferric sulfate (Sigma); about 0.65 μg/mL of Ferric Nitrate (Sigma); about 0.1% (v/v) of CD Lipid Concentrate (Invitrogen); 0.1˜20 μg/mL of Insulin (Baiying, CN); 1-5 mM of Nicotinamide (Sigma); and 0.5˜4% (v/v) of Human Platelet Lysate (PLT, BI).

Composition of CD34A: IMDM (Sigma) (100%); about 1% (v/v) of GlutaMAX-1 (Invitrogen); about 1% (v/v) of NEAA (Invitrogen); 0.1˜20 mg/mL of Bovine serum albumin (BSA, Sigma); 1˜400 μM of Monothioglycerol (MTG); about 80 μg/mL of Ascorbic acid (Sigma); 1˜200 μg/mL of Transferrin (Sigma); 0.1% (v/v) of Trace elements A (Mediatech); 0.1% (v/v) of Trace elements B (Mediatech); 0.1% (v/v) of Trace elements C (Mediatech); about 0.1% (v/v) of CD Lipid Concentrate (Invitrogen).

### Examples 5-7: Novel Cell Basal Medium Promote Cell Differentiation

The present examples demonstrate use of an exemplary NKSFM at the specific stage(s) promotes efficient iNK cell differentiation from hPSCs.

### Example 5

hiPSCs were prepared as described in CN108373998B. On Day −1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising indicated basal medium (see Table 2.1) and supplemented with BMP4 (25 ng/ml), VEGF (50 ng/mL), bFGF (0.5 ng/ml) and CHIR99021 (4 μM) for 2 days. On Day 2, lateral mesoderm differentiation (Stage 1-3) continued for 1 day in a Stage 1-3 differentiation medium comprising indicated basal medium and supplemented with BMP4 (25 ng/mL), VEGF (50 ng/ml) and bFGF (0.5 ng/ml) and CHIR99021 (1 μM). On Day 3, HE specification (Stage 1-4) was initiated by adding a Stage 1-4 differentiation medium comprising indicated basal medium and supplemented with VEGF (50 ng/mL), bFGF (5 ng/mL), XAV939 (5 μM) for 1 day. On Day 4 (the end of Stage 1-4), EBs were dissociated into single cells and stained with KDR-APC antibody, the percentage of KDR⁺ cells were detected by flow cytometry. HP differentiation (Stage 1-5) in was initiated by adding a Stage 1-5 differentiation medium comprising indicated basal medium and supplemented with SCF (10 ng/ml), Flt3L (10 ng/ml), VEGF (15 ng/ml) and bFGF (5 ng/mL) for 3 days. On Day 7 (the end of Stage 1-5), EBs were dissociated into single cells and stained with CD34− APC antibody, the percentage of CD34⁺ HP cells was detected by flow cytometry. To initiate the differentiation from HP cells into iNK cells (Stage 2), EBs were collected and seeded in 6-well-plates coated with matrix proteins, DLL4 and VCAM1, in a Stage 2 differentiation medium comprising NKSFM and supplemented with SCF (20 ng/mL), Flt3L (10 ng/mL), IL-7 (25 ng/mL, Peprotech), IL-3 (5 ng/mL, Peprotech), IL-2 (700 IU/mL), IL-15 (10 ng/mL, Peprotech) for 14 days. On Day 21 (Stage 2-wk2), derived cells were counted and stained with CD56-PE antibody, the percentage of CD56⁺ iNK cells were detected by flow cytometry. By comparing #2 with #1, the results in Table 2.1 demonstrate that the differentiation efficiency (CD34⁺%, CD56⁺% and CD56⁺ cell number) for HE cells, HP cells or iNK cells is improved when conventional IF-4 is replaced with NKSFM during Stage 1-5 (Table 2.1). This improvement is attributed to the addition of NAM, PLT and Heparin sodium in NKSFM as compared with IF-4. Further, by comparing #3 (control experiment) with #2, the results demonstrate that NKSFM is not applicable for Stage 1-2, Stage 1-3 and Stage 1-4 because it causes a very low KDR+%.

TABLE 2.1
Effect of different basal medium during Stage 1 and Stage 2.

| | Basal Medium | | | | | | |
|---|---|---|---|---|---|---|---|
| | Stage 1-2, | | | | | | CD56⁺ cell |
| | Stage 1-3, | | | KDR⁺ % | CD34⁺ % | CD567⁺ % | number (10⁶) |
| # | Stage 1-4 | Stage 1-5 | Stage 2 | Stage 1-4 | Stage 1-5 | Stage 2-wk 2 | Stage 2-wk 2 |
| 1 | IF-4 | IF-4 | NKSFM | 81.43% | 23.06% | 39.30% | 3.62 |
| 2 | IF-4 | NKSFM | NKSFM | 83.34% | 44.21% | 50.34% | 8.53 |

| | | | | | |
|---|---|---|---|---|---|
| 3 | NKSFM | NKSFM | NKSFM | 21.04% | Aborted at Stage 1-4 |
| | | | | | for very low KDR+ % |

### Example 6

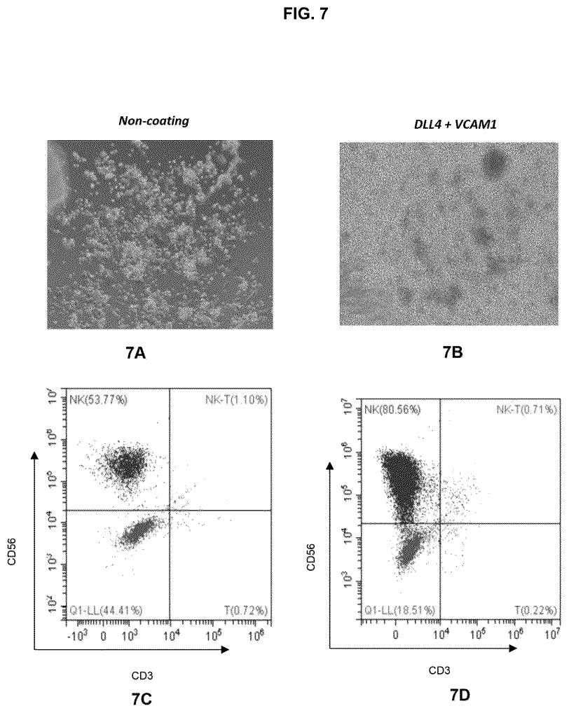

hiPSCs were prepared as described in CN108373998B. Basal medium used during Stage 2 of differentiation was also evaluated. On Day-1, Embryoid body (EB) formation (Stage 1-1) was carried out by placing individualized hiPSCs in E8 medium containing 10 μM Blebbistatin in T25 flasks on a shaker and cultured overnight. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF (15 ng/mL), bFGF (0.5 ng/mL) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/ml), VEGF (15 ng/mL) and bFGF (0.5 ng/mL) and CHIR99021 (4 μM). On Day 3, HE specification (Stage 1-4) was initiated by adding a Stage 1-4 differentiation medium comprising IF-4 basal medium and supplemented with VEGF (15 ng/mL), bFGF (5 ng/ml), SB431542 (6 μM) for 1 day. On Day 4 (the end of Stage 1-4), EBs were dissociated into single cells and stained with KDR-APC antibody, the percentage of KDR+ cells was detected by flow cytometry. HP differentiation (Stage 1-5) in was initiated by adding a Stage 1-5 differentiation medium comprising NKSFM and supplemented with SCF (50 ng/ml), Flt3L (10 ng/mL), VEGF (15 ng/mL) and bFGF (5 ng/mL) for 5 days. To initiate the differentiation from HP cells into iNK cells (Stage 2), EBs of Day 9 were collected and seeded in 24-well-plates coated with matrix proteins, DLL4 and VCAM1, in a Stage 2 differentiation medium comprising indicated basal medium (see Table 2.2) and supplemented with SCF (20 ng/ml), Flt3L (10 ng/ml), IL-7 (25 ng/ml), IL-3 (5 ng/mL), IL-2 (700 IU/mL), IL-15 (10 ng/ml) for 13 days. On Day 22 (Stage 2-wk2), derived cells were counted and stained with CD56-PE antibody, the percentage of CD56+ iNK cells was detected by flow cytometry. FIG. 6 demonstrates that NKSFM used discontinuously or continuously during Stage 2 significantly promoted iNK cell differentiation efficiency (CD56+ cell % (FIG. 6A) and CD56+ cell number (FIG. 6B)) as compared with the other basal media used during Stage 2.

TABLE 2.2
Basal media used during Stage 2.

| | Basal Medium | | | |
|---|---|---|---|---|
| | Stage 1-2, Stage 1-3, | | Stage | Stage |
| # | Stage 1-4 | Stage 1-5 | 2-wk 1 | 2-wk 2 |
| 1 | IF-4 | NKSFM | IF-4 | IF-4 |
| 2 | IF-4 | NKSFM | NKM | NKM |
| 3 | IF-4 | NKSFM | NKSFM-EP | NKSFM-EP |
| 4 | IF-4 | NKSFM | NKSFM | NKSFM |
| 5 | IF-4 | NKSFM | IF-4 | NKM |
| 6 | IF-4 | NKSFM | IF-4 | NKSFM-EP |
| 7 | IF-4 | NKSFM | IF-4 | NKSFM |

### Example 7

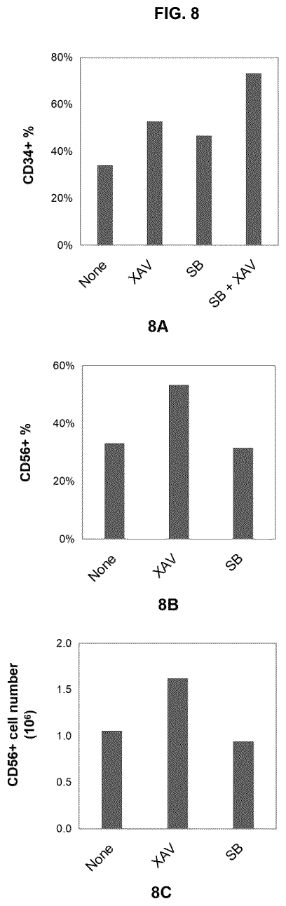

hiPSCs were prepared as described in CN108373998B. The effect of plate coating during Stage 2 on iNK differentiation was further evaluated. On Day-1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising CD34A basal medium and supplemented with BMP4 (25 ng/mL), VEGF (15 ng/mL), bFGF (0.5 ng/ml) and CHIR99021 (4 μM) for 2 days. On Day 2, lateral mesoderm differentiation (Stage 1-3) continued for 1 day in a Stage 1-3 differentiation medium comprising CD34A basal medium and supplemented with BMP4 (25 ng/mL), VEGF (15 ng/mL) and bFGF (0.5 ng/mL) and CHIR99021 (4 μM). On Day 3, HE specification (Stage 1-4) was initiated by adding a Stage 1-4 differentiation medium comprising CD34A basal medium and supplemented with insulin (5 μg/mL), VEGF (15 ng/ml), bFGF (5 ng/ml), SB431542 (6 μM) for 1 day. On Day 4 (the end of Stage 1-4), HP differentiation (Stage 1-5) in was initiated by adding a Stage 1-5 differentiation medium comprising CD34A basal medium and supplemented with SCF (10 ng/ml), Flt3L (10 ng/ml), VEGF (15 ng/mL) and bFGF (5 ng/mL). To initiate the differentiation from HP cells into iNK cells (Stage 2), EBs of Day 11 were collected and seeded in 24-well-plates without matrix or coated with DLL4 and VCAM1, in a Stage 2 differentiation medium comprising NKSFM and supplemented with SCF (20 ng/mL), Flt3L (10 ng/ml), IL-7 (25 ng/ml), IL-3 (5 ng/mL), IL-15 (10 ng/mL) for 14 days. On Day 25 (Stage 2-wk2), derived cells were counted and stained with CD56-PE antibody, the percentage of CD56+ iNK cells was detected by flow cytometry. Results demonstrate that plate coating with DLL4 and VCAM1 during Stage 2 significantly promotes iNK differentiation as compared to iNK differentiation in the absence of plate coating (FIG. 7). The differentiation from hPSCs to immature iNK cells was accomplished in only 26 days. Furthermore, the percentage of CD56+CD3− iNK cells at the end of Stage 2 is as high as 80.56% when differentiation was performed with NKSFM and DLL4/VCAM1.

### Examples 8-10: Wnt Pathway Activation and Inhibition Promote Cell Differentiation

The present examples demonstrate that Wnt activation and inhibition at specific sub-stages during hematopoietic differentiation is crucial for both hematopoietic differentiation and later NK differentiation.

### Example 8

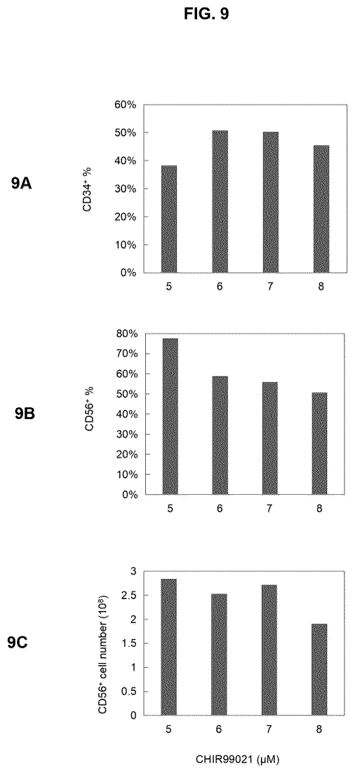

hiPSCs were prepared as described in CN108373998B. On Day −1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising IF-4 basal medium and supplemented with BMP4 (25 ng/ml), VEGF (50 ng/ml), bFGF (0.5 ng/mL) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/ml), VEGF (50 ng/mL) and bFGF (0.5 ng/ml) and CHIR99021 (1 μM). On Day 3, HE specification (Stage 1-4) was initiated by adding a Stage 1-4 differentiation medium comprising IF-4 basal medium and supplemented with VEGF (50 ng/mL), bFGF (5 ng/ml), and without or with XAV939 (5 μM) and/or SB431542 (6 μM) for 1 day. On Day 4, HP differentiation (Stage 1-5) was initiated by adding a Stage 1-5 differentiation medium comprising NKSFM and supplemented with SCF (50 ng/ml), Flt3L (10 ng/ml), VEGF (15 ng/mL) and bFGF (5 ng/mL) for 6 days. On Day 10 (the end of Stage 1), EBs were dissociated into single cells and stained with CD34-APC antibody (BD, #555824), the percentage of CD34⁺ HP cells was detected by flow cytometry. To initiate the differentiation from HP cells into iNK cells (Stage 2), EBs were collected and seeded in 24-well-plates coated with matrix proteins, DLL4 and VCAM1, in a Stage 2 differentiation medium comprising NKSFM and supplemented with SCF (20 ng/ml), Flt3L (10 ng/ml), IL-7 (25 ng/mL), IL-3 (5 ng/mL), IL-2 (700 IU/mL), IL-15 (10 ng/mL) for 15 days. On Day 25 (the end of Stage 2), derived cells were counted and stained with CD56-PE antibody (BD, #555516), the percentage of CD56⁺ iNK cells was detected by flow cytometry. Results of FIG. 8 demonstrate that inhibition of Wnt pathway by XAV939 during HE differentiation (Stage 1-4) significantly promotes the output of both CD34⁺ HP cells (FIG. 8A) and CD56⁺ INK cells (FIGS. 8B and 8C) as compared to the differentiation (control experiment) without both XAV939 and SB431542 or the differentiation with SB431542 only. In addition, as compared to the differentiation (control) without both XAV939 and SB431542, although the differentiation with SB431542 only shows improvement in increasing CD34⁺ HP cell production at a certain degree, it does not show any improvement in the differentiation of iNK cells. Furthermore, use of SB431542 in combination with XAV939 can further promote the output of CD34⁺ HP cells (FIG. 8A).

### Example 9

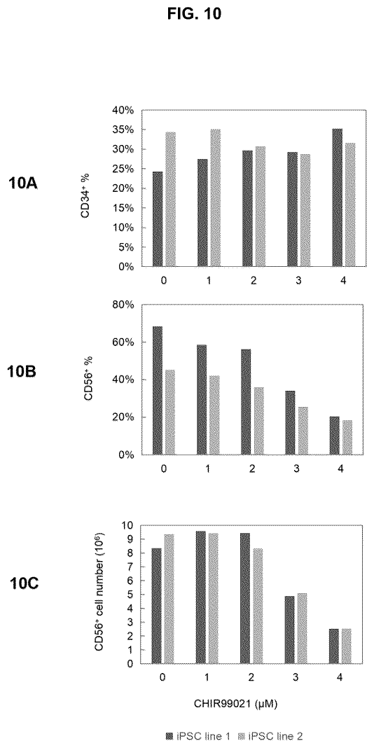

hiPSCs were prepared as described in CN108373998B. The concentration of Wnt signaling pathway activator, CHIR99021, used during Stage 1-2 was optimized. On Day −1, Embryoid body (EB) formation (Stage 1-1) was carried out by placing individualized hiPSCs in E8 medium containing 10 μM Blebbistatin in T25 flasks on a bellydancer and cultured overnight. On Day 0, lateral mesoderm differentiation (Stage 1-2) was initiated by changing the medium to a Stage 1-2 differentiation medium comprising IF-4 basal medium and supplemented with BMP4 (25 ng/ml), VEGF (50 ng/ml), bFGF (0.5 ng/mL, Peprotech) and CHIR99021 at either of 5, 6, 7, or 8 μM for 2 days. On Day 2, lateral mesoderm differentiation (Stage 1-3) continued for 1 day in a Stage 1-3 differentiation medium comprising IF-4 basal medium and supplemented with CHIR99021 (1 μM), BMP4 (25 ng/ml), VEGF (50 ng/mL) and bFGF (0.5 ng/ml). On Day 3, HE specification (Stage 1-4) was initiated by adding a Stage 1-4 differentiation medium comprising IF-4 basal medium and supplemented with VEGF (50 ng/ml), bFGF (5 ng/ml), XAV939 (5 μM) for 1 day. On Day 4, HP differentiation (Stage 1-5) was initiated by adding a Stage 1-5 differentiation medium comprising NKSFM and supplemented with SCF (10 ng/mL), Flt3L (10 ng/ml), VEGF (15 ng/mL) and bFGF (5 ng/mL) for 3 days. On Day 7 (the end of Stage 1), EBs were dissociated into single cells and stained with CD34-APC antibody (BD, #555824), the percentage of CD34⁺ HP cells was detected by flow cytometry. To initiate the differentiation from HP cells into iNK cells (Stage 2), EBs were collected and seeded in T182 flasks coated with matrix proteins, DLL4, in a Stage 2 differentiation medium comprising NKSFM and supplemented with SCF (20 ng/mL), Flt3L (10 ng/ml), IL-7 (25 ng/ml), IL-3 (5 ng/mL), IL-2 (400 IU/mL), IL-15 (10 ng/mL) for 17 days. On Day 24 (the end of Stage 2), cells were counted and stained with CD56-PE antibody (BD, #555516), the percentage of CD56⁺ iNK cells was detected by flow cytometry. Results of FIG. 9 demonstrate that higher concentration of CHIR99021 during Stage 1-2 is generally more advantageous for the differentiation into CD34+HP cells (FIG. 9A), but is somewhat disadvantageous for the differentiation into CD56+ iNK cells (FIGS. 9B and 9C).

### Example 10

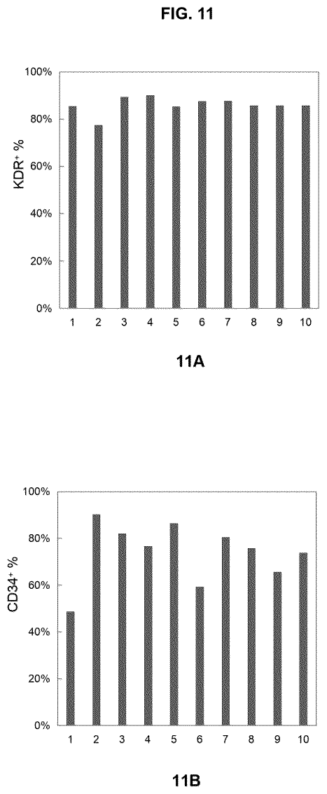

hiPSCs were prepared as described in CN108373998B. The concentration of Wnt signaling pathway activator, CHIR99021, used during Stage 1-3 was optimized. On Day −1, Embryoid body (EB) formation (Stage 1-1) was carried out by placing individualized hiPSCs in E8 medium containing 10 μM Blebbistatin in T25 flasks on a shaker and cultured overnight. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF (50 ng/ml), bFGF (0.5 ng/ml) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/ml), VEGF (50 ng/ml) and bFGF (0.5 ng/ml) and CHIR99021 at either of 0, 1, 2, 3, or 4 μM. On Day 3, HE specification (Stage 1-4) was initiated by adding a Stage 1-4 differentiation medium comprising IF-4 basal medium and supplemented with VEGF (50 ng/mL), bFGF (5 ng/mL), XAV939 (5 μM) for 1 day. On Day 4, HP differentiation (Stage 1-5) was initiated by adding a Stage 1-5 differentiation medium comprising NKSFM and supplemented with SCF (10 ng/ml), Flt3L (10 ng/ml), VEGF (15 ng/mL) and bFGF (5 ng/mL) for 3 days. On Day 7 (the end of Stage 1), EBs were dissociated into single cells and stained with CD34-APC antibody (BD, #555824), the percentage of CD34⁺ HP cells was detected by flow cytometry. To initiate the differentiation from HP cells into iNK cells (Stage 2), EBs were collected and seeded in 6-well-plates coated with matrix proteins, DLL4 and VCAM1, in a Stage 2 differentiation medium comprising NKSFM and supplemented with SCF (20 ng/mL), Flt3L (10 ng/ml), IL-7 (25 ng/ml, Peprotech), IL-3 (5 ng/mL), IL-2 (400 IU/mL), IL-15 (10 ng/mL) for 17 days. On Day 24 (the end of Stage 2), derived cells were counted and stained with CD56-PE antibody (BD, #555516), the percentage of CD56+ iNK cells was detected by flow cytometry. Results of FIG. 10 demonstrate that lower concentration of CHIR99021 during Stage 1-3 is somewhat disadvantageous for the differentiation into CD34+HP cells (FIG. 10A), but is generally more advantageous for the differentiation into CD56+ iNK cells (FIGS. 10B and 10C).

Thus, the present examples demonstrate that higher Wnt signaling activation in the initial stage (Stage 1-2) and lower Wnt signaling activation in the late stage (Stage 1-3) for lateral mesoderm differentiation followed by Wnt signaling inhibition during HE differentiation is crucial for highly efficient differentiation of hPSCs into HE, HP and iNK cells, in particular, HP and iNK cells.

### Examples 11-12: Modulation of the Concentration of VEGF in Stage 1 Promotes Cell Differentiation

The present examples demonstrate that modulation of the concentration of VEGF during Stage 1 of differentiation is beneficial for efficient differentiation of hPSCs into HE, HP or iNK cells.

### Example 11

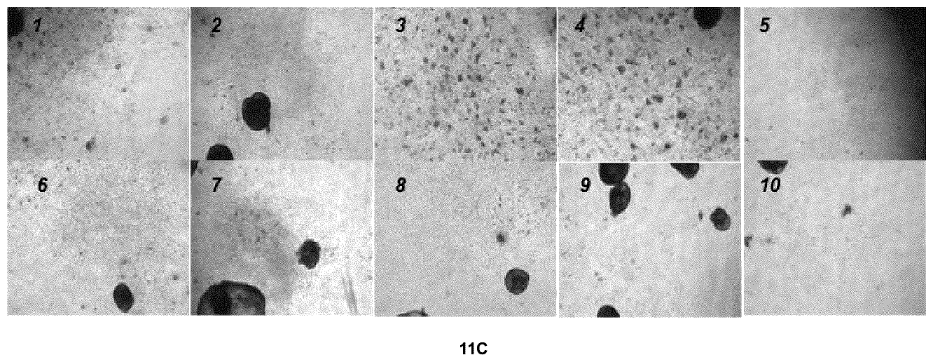

hiPSCs were prepared as described in CN108373998B. The effect of different VEGF concentrations in the substages of the differentiation of hPSCs on the HE or HP cells was evaluated. On Day −1, Embryoid body (EB) formation (Stage 1-1) was carried out by placing individualized hiPSCs in E8 medium containing 10 μM Blebbistatin in T25 flasks on a shaker and cultured overnight. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF with the concentration as shown in Table 3.1, bFGF (0.5 ng/mL) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/mL), VEGF with the concentration as shown in Table 3.1 and bFGF (0.5 ng/ml) and CHIR99021 (1 μM). On Day 3, HE differentiation (Stage 1-4) was initiated by adding a Stage 1-4 differentiation medium comprising IF-4 basal medium and supplemented with VEGF with the concentration as shown in Table 3.1, bFGF (5 ng/ml), XAV939 (5 μM) and SB431542 (6 μM) for 1 day. On Day 4 (the end of Stage 1-4), EBs were dissociated into single cells and stained with KDR-APC antibody (BD, #560495), the percentage of KDR⁺ cells was detected by flow cytometry. HP differentiation (Stage 1-5) was initiated by adding a Stage 1-5 differentiation medium comprising NKSFM and supplemented with SCF (10 ng/mL), Flt3L (10 ng/ml), VEGF with the concentration as shown in Table 3.1 and bFGF (5 ng/mL) for 6 days. On Day 10 (the end of Stage 1), EBs were dissociated into single cells and stained with CD34-APC antibody (BD, #555824), the percentage of CD34⁺ HP cells was detected by flow cytometry. To initiate the differentiation from HP cells into iNK cells (Stage 2), EBs were collected and seeded in 24-well-plates coated with matrix proteins, DLL4 and VCAM1, in a Stage 2 differentiation medium comprising NKSFM and supplemented with SCF (20 ng/ml), Flt3L (10 ng/ml), IL-7 (25 ng/ml), IL-3 (5 ng/ml), IL-2 (700 IU/mL), IL-15 (10 ng/ml) for 14 days. On Day 24 (the end of Stage 2), morphology of iNK cells under different conditions was recorded as shown in FIG. 11C. As shown in FIGS. 11A and 11B, different VEGF concentration shows minimal effects on the percentage of KDR+ HE cells, but significantly impacts on the percentage of CD34+ HP cells and the efficiency of further differentiation into iNK cells. In detail, higher VEGF concentration during Stage 1-2 and Stage 1-3 is generally beneficial for the differentiation into HP cells and iNK cells, and higher VEGF concentration during Stage 1-5 is generally beneficial for the differentiation into HP cells, but not into iNK cells.

TABLE 3.1
Effects of modulation of VEGF concentrations
during Stage 1 on the HE and HP cells.

| | VEGF (ng/mL) | | |
|---|---|---|---|
| # | Stage 1-2, Stage 1-3 | Stage 1-4 | Stage 1-5 |
| 1 | 15 | 15 | 15 |
| 2 | 25 | 25 | 25 |
| 3 | 50 | 50 | 50 |
| 4 | 100 | 100 | 100 |
| 5 | 15 | 25 | 25 |
| 6 | 15 | 50 | 50 |
| 7 | 15 | 100 | 100 |
| 8 | 15 | 15 | 25 |
| 9 | 15 | 15 | 50 |
| 10 | 15 | 15 | 100 |

### Example 12

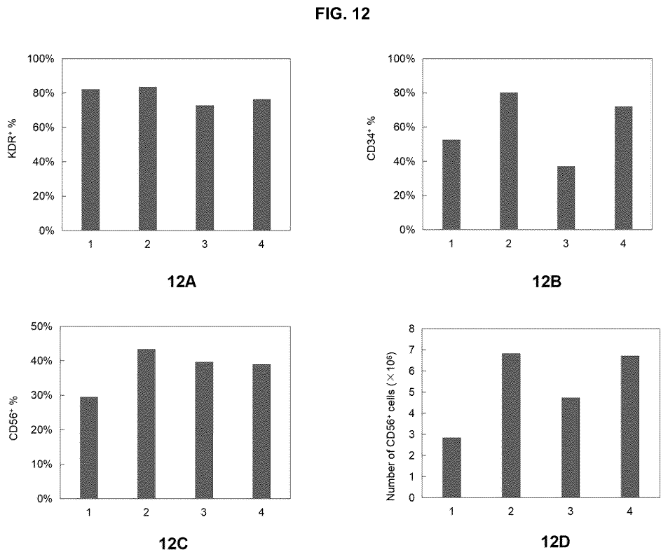

hiPSCs were prepared as described in CN108373998B. The effects of different VEGF concentrations in the substages of the differentiation of hPSCs on the HE, HP and iNK cells were evaluated. On Day −1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF with the concentration as shown in Table 3.2, bFGF (0.5 ng/mL) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/mL), VEGF with the concentration as shown in Table 3.2 and bFGF (0.5 ng/ml) and CHIR99021 (1 μM). On Day 3, HE specification (Stage 1-4) was initiated by adding a Stage 1-4 differentiation medium comprising IF-4 basal medium and supplemented with VEGF with the concentration as shown in Table 3.2, bFGF (5 ng/ml), XAV939 (5 μM) for 1 day. On Day 4 (the end of Stage 1-4), EBs were dissociated into single cells and stained with KDR-APC antibody (BD, #560495), the percentage of KDR+ cells was detected by flow cytometry. HP differentiation (Stage 1-5) was initiated by adding a Stage 1-5 differentiation medium comprising NKSFM and supplemented with SCF (10 ng/mL), Flt3L (10 ng/mL), VEGF with the concentration as shown in Table 3.2 and bFGF (5 ng/mL) for 3 days. On Day 7 (the end of Stage 1), EBs were dissociated into single cells and stained with CD34-APC antibody (BD, #555824), the percentage of CD34+HP cells was detected by flow cytometry. To initiate the differentiation from HP cells into iNK cells (Stage 2), EBs were collected and seeded in 24-well-plates coated with matrix proteins, DLL4 and VCAM1, in a Stage 2 differentiation medium comprising NKSFM and supplemented with SCF (20 ng/mL), Flt3L (10 ng/ml), IL-7 (25 ng/ml), IL-3 (5 ng/ml), IL-2 (700 IU/mL), IL-15 (10 ng/mL) for 14 days. On Day 21 (the end of Stage 2), derived cells were counted and stained with CD56-PE antibody (BD, #555516), the percentage of CD56+ iNK cells was analyzed by flow cytometry. Results of FIG. 12 show that higher concentration of VEGF during Stage 1, especially during differentiation from hPSCs to HE cells (Stage 1-2, Stage 1-3 and Stage 1-4), significantly improves both HP and iNK cell differentiation.

TABLE 3.2
Effect of modulation of VEGF concentrations
during Stage 1 on the HE, HP and iNK cells.

| | VEGF (ng/mL) | | |
|---|---|---|---|
| # | Stage 1-2, Stage 1-3 | Stage 1-4 | Stage 1-5 |
| 1 | 15 | 15 | 15 |
| 2 | 50 | 50 | 50 |
| 3 | 50 | 15 | 15 |
| 4 | 50 | 50 | 15 |

### Examples 13-14: Expansion and Maturation of Immature iNK Cells by NKSFM

The present examples demonstrate that use of NKSFM can promote the expansion and maturation of NK cells, in particular, immature iNK cells.

### Example 13

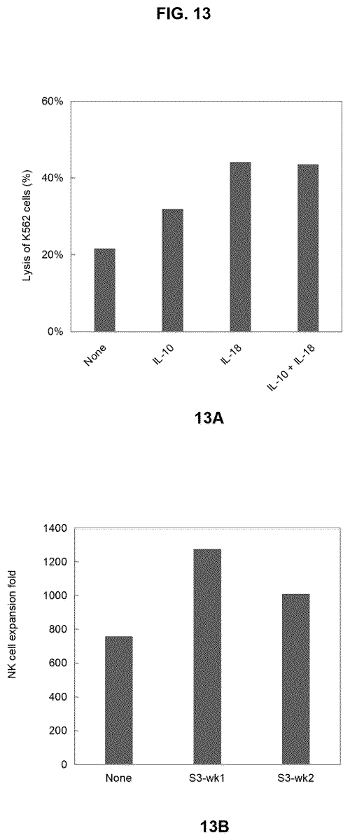

hiPSCs were prepared as described in CN108373998B. On Day −1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF (15 ng/ml), bFGF (0.5 ng/mL) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/mL), VEGF (50 ng/mL) and bFGF (0.5 ng/mL) and CHIR99021 (1 μM). On Day 3, HE specification (Stage 1-4) was initiated by adding a Stage 1-4 differentiation medium comprising IF-4 basal medium and supplemented with VEGF (15 ng/ml), bFGF (5 ng/ml), XAV939 (5 μM) for 1 day. On Day 4 (the end of Stage 1-4), HP differentiation (Stage 1-5) in was initiated by adding a Stage 1-5 differentiation medium comprising IF-4 basal medium and supplemented with SCF (10 ng/ml), Flt3L (10 ng/ml), VEGF (15 ng/mL) and bFGF (5 ng/mL). To initiate the differentiation from HP cells into iNK cells (Stage 2), EBs of Day 10 were collected and seeded in 24-well-plates coated with DLL4 and/or VCAM1, in a Stage 2 differentiation medium comprising NKSFM and supplemented with SCF (20 ng/ml), Flt3L (10 ng/mL), IL-7 (25 ng/mL), IL-3 (5 ng/ml), IL-2 (700 IU/mL), IL-15 (10 ng/ml, Peprotech) for 14 days. On D24 (the end of Stage 2), immature iNK cells were cyropreserved in liquid nitrogen at 50×10⁶ cells/mL in a Multiple Electrolytes injection (Zhendong Health, H20113035) supplemented with HSA (40 mg/mL) and DMSO (10%). Thawed D24 iNK cells were co-cultured with feeder cells (Nuwacell, Feeder cells:INK cells=1:1) in 6-well-plates in a Stage 3 expansion and maturation medium comprising a NKSFM and supplemented with or without one or more reagents selected from IL-10 (20 ng/ml), and IL-18 (50 ng/mL). The feeder cells are K562 cells (Procell) genetically engineered to co-express mbIL-21 (membrane bound IL-21), 41BBL, CD19, and CD64. Feeder cells were inactivated by gamma irradiation before co-culture with iNK cells. On Day 11 of co-culture, iNK cells were collected and stained with CD56-PE antibody (BD, #555516), the percentage of CD56+ cells was analyzed by flow cytometry. The cytotoxicity of Stage 3 iNK cells towards K562 cells was detected by CFSE/7AAD assay after 4 hr co-culture at E:T of 3:1. As shown in FIG. 13A, IL-10 (20 ng/mL), and/or IL-18, in particular, IL-18 can significantly improve the cytotoxicity of iNK cells.

### Example 14

hiPSCs were prepared as described in CN108373998B. On Day −1, Embryoid body (EB) formation (Stage 1-1) was carried out by placing individualized hiPSCs in E8 medium containing 10 μM Blebbistatin in T25 flasks on a bellydancer and culture overnight. On Day 0, lateral mesoderm differentiation (Stage 1-2) was initiated by changing the medium to a Stage 1-2 differentiation medium comprising IF-4 basal medium and supplemented with BMP4 (25 ng/ml), VEGF (15 ng/ml), bFGF (0.5 ng/ml) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/ml), VEGF (15 ng/mL) and bFGF (0.5 ng/mL) and CHIR99021 (1 μM). On Day 3, HE specification (Stage 1-4) was initiated by adding a Stage 1-4 differentiation medium comprising IF-4 basal medium and supplemented with VEGF (15 ng/ml), bFGF (5 ng/ml), XAV939 (5 μM) for 1 day. On Day 4 (the end of Stage 1-4), HP differentiation (Stage 1-5) in was initiated by adding a Stage 1-5 differentiation medium comprising IF-4 basal medium and supplemented with SCF (10 ng/ml), Flt3L (10 ng/ml), VEGF (15 ng/ml) and bFGF (5 ng/mL). To initiate the differentiation from HP cells into iNK cells (Stage 2), EBs of Day 10 were collected and seeded in 24-well-plates coated with DLL4 and VCAM1, in a Stage 2 differentiation medium comprising NKSFM and supplemented with SCF (20 ng/ml), Flt3L (10 ng/mL), IL-7 (25 ng/ml), IL-3 (5 ng/ml), IL-2 (700 IU/mL), IL-15 (10 ng/ml) for 14 days. On D24 (the end of Stage 2), immature iNK cells were cyropreserved in liquid nitrogen at 50×10⁶ cells/mL in Dextran injection (SJZ No. 4 Pharmaceutical, H13022493) supplemented with HSA (40 mg/mL) and DMSO (10%). Thawed D25 iNK cells were co-cultured with feeder cells (Feeder cells:INK cells=0.5:1) in 6-well-plates in a Stage 3 expansion and maturation medium comprising a NKSFM basal medium and supplemented with IL-2 (100 IU/mL) and with or without IL-18 (10-50 ng/mL, added during S3-wk1 or S3-wk2 as indicated) for 2 weeks. On Day 14 of co-culture, iNK cells were collected and stained with CD56-PE antibody (BD, #555516), and the percentage of CD56+ cells was analyzed by flow cytometry. As shown in FIG. 13B, the combination of IL-18 with IL-2, in particular, the combination of IL-18 added during the initial period of the Stage 3 with IL-2, can improve the expansion fold of iNK cells as compare with IL-2 only.

### Examples 15-20: Exemplary Characterization of Hematopoietic Lineage Cells Formed During the Process of Producing iNK Cells from hPSCs

### Example 15

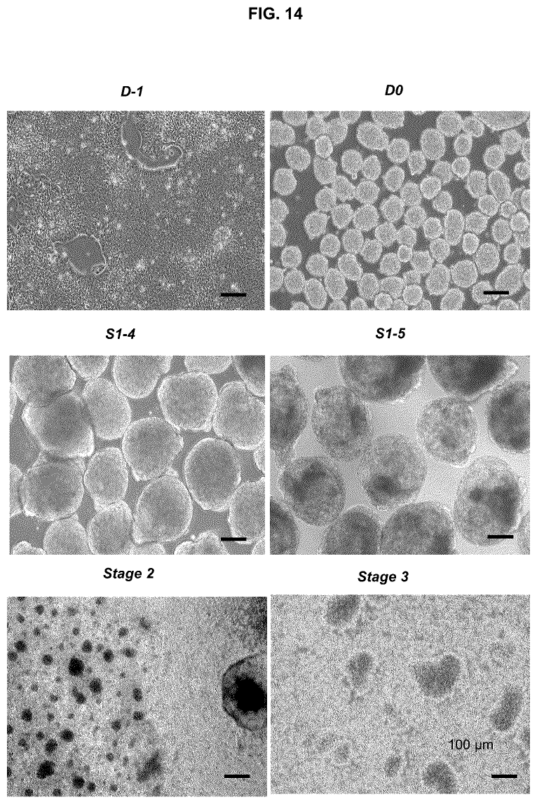

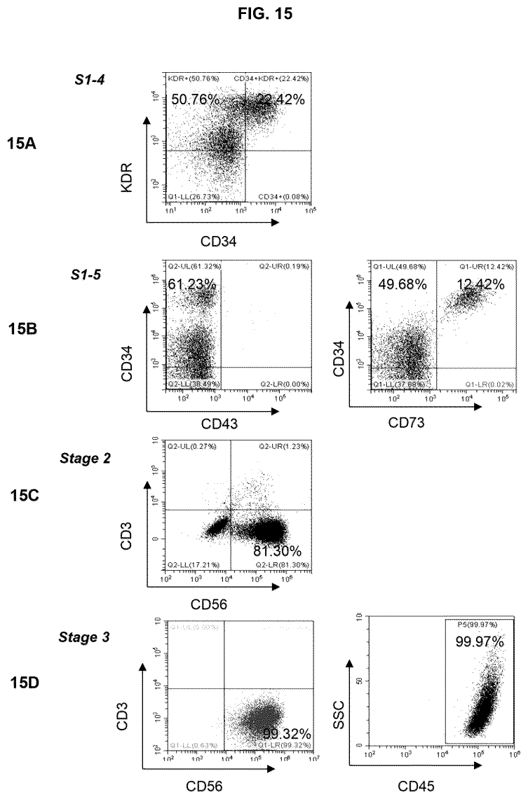

hiPSCs were prepared as described in CN108373998B. On Day −1, Embryoid body (EB) formation (Stage 1-1) was carried out by placing individualized hiPSCs in E8 medium containing 10 μM Blebbistatin in T25 flasks on a bellydancer and culture overnight. D-1 and DO cells were analyzed by Microscopy. On Day 0, lateral mesoderm differentiation (Stage 1-2) was initiated by changing the medium to a Stage 1-2 differentiation medium comprising IF-4 basal medium and supplemented with BMP4 (25 ng/mL), VEGF (50 ng/ml), bFGF (0.5 ng/ml) and CHIR99021 (4 μM) for 2 days. On Day 2, lateral mesoderm differentiation (Stage 1-3) continued for 1 day in a Stage 1-3 differentiation medium comprising IF-4 basal medium and supplemented with BMP4 (25 ng/ml), VEGF (50 ng/ml) and bFGF (0.5 ng/mL) and CHIR99021 (1 μM). On Day 3, HE specification (Stage 1-4) was initiated by adding a Stage 1-4 differentiation medium comprising IF-4 basal medium and supplemented with VEGF (50 ng/ml), bFGF (5 ng/ml), XAV939 (5 μM) for 1 day. At the end of Stage 1-4, cells were analyzed by Microscopy and FACS. On Day 4 (the end of Stage 1-4), HP differentiation (Stage 1-5) in was initiated by adding a Stage 1-5 differentiation medium comprising a NKSFM and supplemented with SCF (10 ng/ml), Flt3L (10 ng/ml), VEGF (15 ng/mL) and bFGF (5 ng/mL). At the end of Stage 1-5, cells were analyzed by Microscopy and FACS. To initiate the differentiation from HP cells into iNK cells (Stage 2), EBs of Day 7 were collected and seeded in 24-well-plates coated with DLL4 and VCAM1, in a Stage 2 differentiation medium comprising a NKSFM and supplemented with SCF (20 ng/ml), Flt3L (10 ng/mL), IL-7 (25 ng/ml), IL-3 (5 ng/ml), IL-2 (700 IU/mL), IL-15 (10 ng/ml) for 17 days. At the end of Stage 2, cells were analyzed by Microscopy and FACS. On D24 (the end of Stage 2), immature iNK cells were cyropreserved in liquid nitrogen at 50×10⁶ cells/mL in Dextran injection (SJZ No. 4 Pharmaceutical, H13022493) supplemented with HSA (40 mg/mL) and DMSO (10%). Thawed D24 iNK cells were co-cultured with feeder cells (Feeder cells:INK cells=1:1) in 6-well-plates in a Stage 3 expansion and maturation medium comprising a NKSFM basal medium and supplemented with IL-2 (100 IU/mL) and IL-18 (10 ng/mL). On Day 11 of co-culture, iNK cells were collected and stained with CD56-PE antibody (BD, #555516), CD56+ cells were analyzed by Microscopy and FACS. FIG. 14 shows the morphologies of hematopoietic lineage cells formed during the different differentiation stages of producing iNK cells from hPSCs. Results showed that the EB size increased gradually during Stage 1 of iNK cell differentiation. After EB plating, iNK cells formed small cell clusters around attached EBs during Stage 2. During Stage 3, INK cells proliferated vigorously and were prone to gather into cell clumps in static conditions. As shown in FIG. 15A, at the end of Stage 1-4, the cells show efficient lateral mesoderm and HE differentiation. As shown in FIG. 15B, at the end of Stage 1-5, the percentage of CD34+CD43− HP cells is as high as 61.23%, which demonstrates efficient HP differentiation. As shown in FIG. 15C, at the end of Stage 2, the percentage of CD56+CD3− iNK cells is as high as 81.30%, which demonstrates efficient iNK cell specification. As shown in FIG. 15D, at the end of Stage 3, the percentage of CD56+CD3− iNK cells is as high as 99.32%, which demonstrates that highly pure iNK cells are obtained.

### Example 16

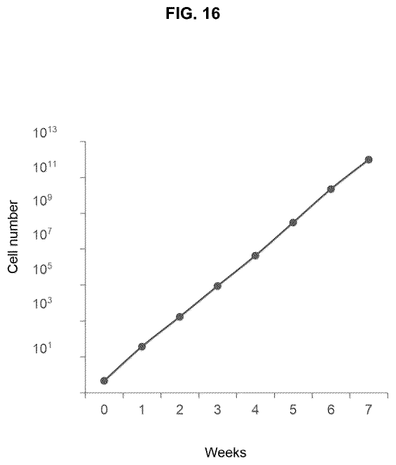

In order to test the proliferation potential of our iNK cells, immature iNK cells obtained from Stage 2 in the Example 15 were co-cultured with feeder cells (Nuwacell) (Feeder cells:iNK cells=1:1) in 6-well-plates in a Stage 3 expansion and maturation medium comprising a NKSFM basal medium and supplemented with IL-2 (100 IU/mL) and IL-18 (10 ng/mL). iNK cells were continuously expanded for 7 weeks with weekly feeder cell-stimulation. The result shows that iNK cells obtained by this method is of high proliferation potential and can be repeatedly stimulated for continuous expansion for at least 7 weeks (FIG. 16). The expansion fold observed herein was much higher compared to hPSC-derived NK cells repeatedly stimulated by aAPCs (mbIL-21 artificial antigen-presenting cells) for continuous long-term expansion from other groups (Knorr D A et al., Stem Cells Transl Med. 2013 April; 2 (4): 274-283). Thus, the resulting immature iNK cells demonstrate high expansion potential.

### Example 17

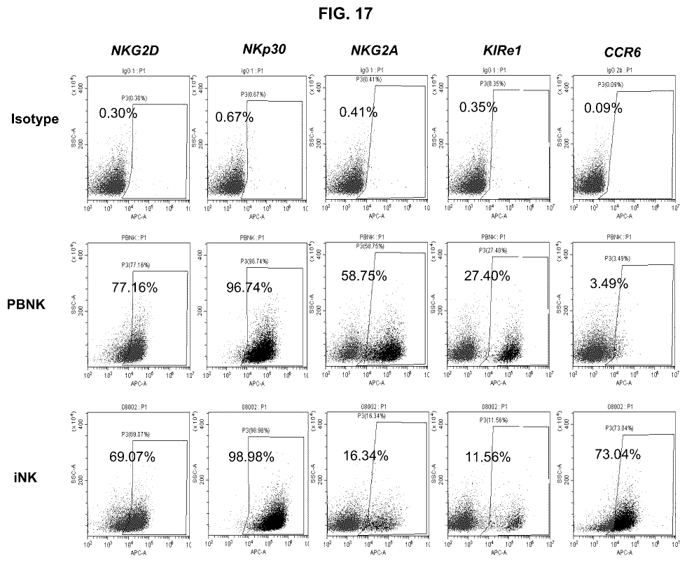

In order to compare the phenotypes of primary NK cells and our iNK cells, the expressions of NK cell surface receptors on PBNK cells and iNK cells obtained in the Example 15 were analyzed by flow cytometry and compared. The Stage 3 iNK cells were stained with CD56-PE (BD, #555516), NKG2D-APC (BD, #558071), Nkp30-APC (BD, #558408), NKG2A-APC (Biolegend, #375107), KIRe1-APC (Biolegend, ##312716) and CCR6-APC (Biolegend, #353416) antibodies. The percentages of NKG2D+, NKp30+, NKG2A+, KIRe1+ and CCR6+ cells in CD56+ population were analyzed by flow cytometry. As shown in FIG. 17, the expressions of activating receptors, NKG2D and NKp30, were comparable in PBNK cells and the INK cells. However, the iNK cells had much lower expression of the inhibitory receptors, NKG2A and KIRe1, as compared to PBNK cells, which is indicative of less inhibition from target cells. Also, the iNK cells had much higher expression of the chemokine receptor, CCR6, as compared to PBNK cells, which is indicative of improved homing to tissues and targets (FIG. 17). Interestingly, the cell surface marker expression on the iNK cells produced with the method described herein was also different from those in the literatures. Thus, the iNK cells demonstrated different expression patterns of surface receptors compared to primary NK cells, which is indicative of greatly improved function.

### Example 18

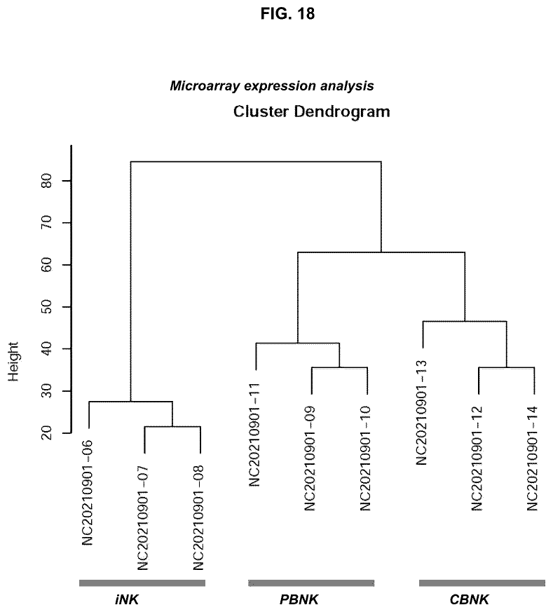

In order to analyze the correlation between primary NK cells and our iNK cells, global gene expressions for Stage 3 iNK cells obtained in the Example 17, PBNK cells, and CBNK cells were assessed by microarray and dendrogram clustering analysis. The results demonstrate that the iNK cells are similar to primary NK cells, but also represent an independent cell type different from both PBNK cells and CBNK cells. Accordingly, the iNK cells are similar to primary NK cells in gene expression, but cluster to a unique cell type (FIG. 18).

### Example 19

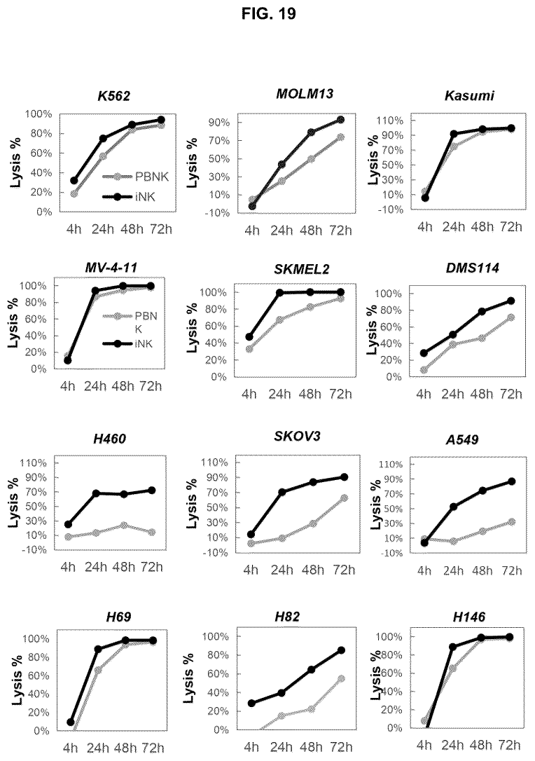

In order to assess the cytolytic function of the iNK cells compared to PBNK cells, the iNK cells or PBNK cells were co-cultured with different types of tumor cells and analyzed for their cytotoxicity. Cryopreserved Stage 3 iNK cells obtained from the Example 17 were thawed for cytotoxicity assays. Different tumor cell lines were labelled with CFSE prior to co-culture with NK cells at E:T of 3:1 (E:T was the ratio of effector cell number to target cell number, wherein the effector cells were the iNK cells or PBNK cells and the target cells were tumor cells). After incubation for the indicated time, cells were stained with 7-AAD (Live/dead staining) and analyzed with flow cytometry. Compared to PBNK cells, the iNK cells show a comparable cytotoxicity towards K562 cells, Kasumi cells, MV-4-11 cells, H69 cells and H146 cells, and much higher cytotoxicity towards H460 cells, SKOV3 cells, A549 cells, SKMEL2 cells, DMS114 cells, MOLM13 cells, and H82 cells. The results indicate that the iNK cells are cytotoxic to a wide range of tumor cell lines, with comparable or improved activity compared to primary NK cells (FIG. 19).

### Example 20

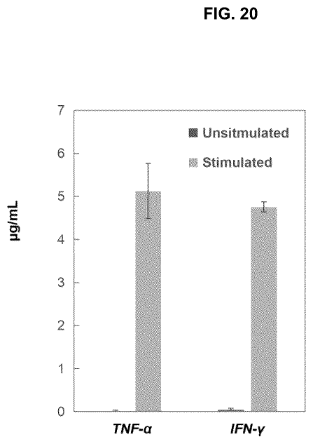

In order to further assess the function of the iNK cells, the iNK cells obtained in the Example 17 were stimulated with 50 ng/ml of phorbol myristate acetate (PMA) and 1 μg/mL of Ionomycin for 4 hours and analyzed for the production of TNF-α and IFN-γ by enzyme-linked immunosorbent assay (ELISA) assay. The result demonstrates that the iNK cells can respond to stimulation to secrete high level of pro-inflammatory cytokines: TNF-α and IFN-γ (FIG. 20).

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure is not to be limited in terms of the particular embodiments described in this application, which are intended as single illustrations of individual aspects of the disclosure. All the various embodiments of the present disclosure will not be described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for promoting the directed differentiation of pluripotent stem cells (PSCs), comprising the steps of:
contacting the PSCs with a maintenance culture medium to form embryoid bodies (EBs);
contacting the EBs with a first differentiation culture medium supplemented with a Wnt signaling pathway activator, or with a first differentiation culture medium supplemented with a Wnt signaling pathway activator and a second differentiation culture medium supplemented with a Wnt signaling pathway activator sequentially, to form mesodermal cells; and
contacting the mesodermal cells with a third differentiation culture medium supplemented with a Wnt signaling pathway inhibitor to obtain hemogenic endothelial (HE) cells.

2. The method of claim 1, further comprising the step of contacting the HE cells with a fourth differentiation culture medium to obtain hematopoietic progenitor (HP) cells.

3. The method of claim 2, further comprising the step of contacting the HP cells with a fifth differentiation culture medium to obtain immature iNK cells.

4. The method of claim 1, wherein the EBs are contacted with the first differentiation culture medium and the second differentiation culture medium sequentially to form mesodermal cells, wherein the Wnt signaling pathway activator in the second differentiation culture medium may be same or different and has an equal or lower concentration as compared to the Wnt signaling pathway activator in the first differentiation culture medium.

5. The method of claim 4, wherein the second differentiation culture medium has the same composition as that of the first differentiation culture medium except that the concentration of the Wnt signaling pathway activator in the second differentiation culture medium is lower than the concentration of the Wnt signaling pathway activator in the first differentiation culture medium.

6. The method of claim 4, wherein the concentration of the Wnt signaling pathway activator in the second differentiation culture medium is from 0 to 4 μM, and the concentration of the Wnt signaling pathway activator in the first differentiation culture medium is from 4 to 8 μM.

7. The method of claim 1, wherein the concentration of the Wnt signaling pathway inhibitor in the third differentiation culture medium is from 1 to 30 μM.

8. The method of claim 1, wherein the Wnt signaling pathway activator is selected from the group consisting of Kenpaullone, 1-Azakenpaullone, CHIR99021, CHIR98014, NP031112, TWS119, AZD2858, AZD1080, SB415286, LY2090314, AR-A014418, SB216763, AR-A014418, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, GF109203X, and any combination thereof.

9. The method of claim 1, wherein the Wnt signaling pathway inhibitor is selected from the group consisting of iCRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, Isoquercitrin, Gigantol, JW55, MSAB, IWR-1-endo, FH535, WIKI4, CCT251545, KYA1797K, NCB-0846, iCRT14, Adavivint, M435-1279, XAV939, and any combination thereof.

10. The method of claim 2, wherein the third differentiation culture medium is further supplemented with a TGF-β signaling pathway inhibitor.

11. The method of claim 2, wherein the fourth differentiation culture medium comprises a basal medium supplemented with the combination of (i) a nicotinamide-based compound, (ii) a heparin-based compound, and (iii) a human platelet lysate.

12. The method of claim 11, wherein the concentration of the nicotinamide-based compound in the fourth differentiation culture medium is from 0.5 to 20 mM.

13. The method of claim 11, wherein the concentration of the heparin-based compound in the fourth differentiation culture medium is from 0.1 to 100 g/mL.

14. The method of claim 11, wherein the concentration of the human platelet lysate in the fourth differentiation culture medium is from 0.1% to 20% by volume.

15. The method of claim 11, wherein the nicotinamide-based compound comprises nicotinamide, and the heparin-based compound comprises heparin sodium.

16. The method of claim 15, wherein the basal medium supplemented with the combination of (i) nicotinamide-based compound, (ii) heparin-based compound, and (iii) human platelet lysate contains IF-4 basal medium in addition to the combination of (i) nicotinamide, (ii) heparin sodium, and (iii) human platelet lysate.

17. The method of claim 15, wherein the basal medium supplemented with the combination of (i) nicotinamide-based compound, (ii) heparin-based compound, and (iii) human platelet lysate comprises a NKSFM basal medium.

18. The method of claim 1, wherein the first to third differentiation basal media comprise the same basal medium.

19. The method of claim 2, wherein the first to fourth differentiation culture media are each independently further supplemented with a VEGF at a concentration of 15 to 100 ng/mL.

20. The method of claim 3, wherein the first to fifth differentiation culture media are chemically defined serum-free and xeno-free differentiation culture media.

21. The method of claim 3, wherein the method is carried out under 3D culture condition.

22. A culture medium for promoting the directed differentiation of pluripotent stem cells (PSCs) into hematopoietic lineage cells, comprising a basal medium and supplemented with a Wnt signaling pathway inhibitor.

23. The culture medium of claim 22, wherein the hematopoietic lineage cells comprise HE cells, HP cells or immature iNK cells.

24. The culture medium of claim 22, wherein the Wnt signaling pathway inhibitor is selected from the group consisting of iCRT3, IWP-O1, IWP-2, IWP-3, IWP-4, Ciclopirox, Cardamonin, Diethyl benzylphosphonate, Disodium Pamidronate Hydrate, Ginsenoside Rh4, KY-05009, Isoquercitrin, Gigantol, JW55, MSAB, IWR-1-endo, FH535, WIKI4, CCT251545, KYA1797K, NCB-0846, iCRT14, Adavivint, M435-1279, XAV939, and any combination thereof.

25. The culture medium of claim 22, wherein the concentration of the Wnt signaling pathway inhibitor in the culture medium is from 1 to 30 μM.

26. The culture medium of claim 22, wherein the culture medium is further supplemented with a TGF-β signaling pathway inhibitor.

27. The culture medium of claim 22, wherein the culture medium is further supplemented with a VEGF at a concentration of 15 to 100 ng/mL.

28. The culture medium of claim 22, wherein the culture medium is a chemically defined serum-free and xeno-free differentiation culture medium.

29. A kit, comprising the culture medium of claim 22.

30. The kit of claim 29, further comprising a first differentiation culture medium-supplemented with a Wnt signaling pathway activator.

31. The kit of claim 30, further comprising a second differentiation culture medium supplemented with a Wnt signaling pathway activator, wherein the Wnt signaling pathway activator in the second differentiation culture medium may be same or different and has an equal or lower concentration as compared to the Wnt signaling pathway activator in the first differentiation culture medium.

32. The kit of claim 31, wherein the second differentiation culture medium has the same composition as that of the first differentiation culture medium except that the concentration of the Wnt signaling pathway activator in the second differentiation culture medium is lower than the concentration of the Wnt signaling pathway activator in the first differentiation culture medium.

33. The kit of claim 31, wherein the concentration of the Wnt signaling pathway activator in the second differentiation culture medium is from 0 to 4 μM, and the concentration of the Wnt signaling pathway activator in the first differentiation culture medium is from 4 to 8 μM.

34. The kit of claim 33, wherein the Wnt signaling pathway activators in the first and second differentiation culture media are each independently selected from the group consisting of Kenpaullone, 1-Azakenpaullone, CHIR99021, CHIR98014, NP031112, TWS119, AZD2858, AZD1080, SB415286, LY2090314, AR-A014418, SB216763, AR-A014418, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, GF109203X, and any combination thereof.

35. The kit of claim 31, wherein the first and second differentiation culture media are each further supplemented with a VEGF at a concentration of 15-100 ng/mL.

36. The kit of claim 31, wherein all culture media in the kit are chemically defined serum-free and xeno-free differentiation culture media comprising the same basal medium.

37. A cell population produced by the method of claim 1.

38. A cell population produced by the method of claim 2.

39. A cell population produced by the method of claim 3.
